(19) United States
(12) Patent Application Publication
(10) Pub. No.: US 2020/0101034 A1
(43) Pub. Date: Apr. 2, 2020

(54) RAPID AND CONTROLLED DELIVERY OF COMPOSITIONS WITH RESTORED ENTOURAGE EFFECTS

(71) Applicant: RECEPTOR HOLDINGS, INC., Seattle, WA (US)

(72) Inventors: Andrea Leone-Bay, Ridgefield, CT (US); Gregory Wesner, Bainbridge Island, WA (US)

(73) Assignee: RECEPTOR HOLDINGS, INC., Seattle, WA (US)

(21) Appl. No.: 16/496,849

(22) PCT Filed: Mar. 23, 2018
(86) PCT No.: PCT/US18/24188

§ 371 Date: Sep. 23, 2019

Related U.S. Application Data
(60) Provisional application No. 62/475,763, filed on Mar. 23, 2017.

(51) Int. Cl. A61K 31/20; A61K 31/352; A61K 31/047; A61K 9/00; A61K 45/06; A23L 33/105; A23L 33/155; A23L 33/16

(52) U.S. Cl. CPC A61K 31/20; A61K 31/352; A61K 31/047; A61K 9/0053; A23V 2002/00; A61K 9/0056; A23L 33/105; A23L 33/155; A23L 33/16; A61K 45/06

(57) ABSTRACT

Fast-acting oral formulations with restored entourage effects are described. The formulations include beneficial combinations of plant-derived molecules to provide restored entourage effects, and one or more carriers. The carriers can include N-acylated fatty amino acids, absorption enhancers, and/or various other beneficial carriers. The fast-acting oral formulations can create administration benefits.

### CROSS REFERENCE TO RELATED APPLICATION

This application claims priority to 62/475,763 filed on Mar. 23, 2017, which is incorporated herein by reference in its entirety as if fully set forth herein.

### FIELD OF THE DISCLOSURE

The current disclosure provides fast-acting oral formulations of plant-based compositions with restored entourage effects. The plant-based compositions include combinations of beneficial plant-derived molecules to provide restored entourage effects. The formulations include one or more carriers, which allow rapid and controlled delivery of the plant-based compositions. The carriers can include N-acylated fatty amino acids, absorption enhancers, and/or various other beneficial carriers.

### BACKGROUND OF THE DISCLOSURE

Historically, the plant world has been the most important source of medicinal agents for the treatment of human and animal disease, and for use as preventative agents and nutritional supplements for maintaining good health. However, for at least the last 150 years, Western medicine has been dominated by synthetic chemical agents.

It is now being increasingly recognized, however, that in certain situations plants and plant extracts are preferable over synthetic chemical agents. A single plant can possess a large number of physiologically active phytochemicals, and extracts containing multiple phytochemicals can exert their effects on a variety of physiologic processes. This variety and combination of physiological effects is not readily replicated by use of singly-created synthetic molecules. In some instances, physiologically active phytochemicals have enhanced activity when present in combination with other physiologically active phytochemicals. The benefits that can arise from the use of whole plant extracts is one of the major tenets of the field of herbal medicine.

One example of the benefits of combined phytochemicals is the synergy in the antioxidant potential of extracts from skin, juice, and seeds of grapes. The pooled extracts have greater antioxidant potential than the individual extracts (Epps et al., J Food Res. 2013. 2(6):33-47).

Another example of a beneficial combination of phytochemicals relates to the use of two cannabis-derived molecules to treat multiple sclerosis. Sativex® (GW Pharma, Wilshire, United Kingdom) is a 1:1 ratio of two cannabis-derived molecules, delta-9-tetrahydrocannabinol (Δ⁹-THC or THC) and cannabidiol (CBD); and is approved in several countries to treat the spasticity and neuropathy associated with multiple-sclerosis (Syed et al., Drug 2014. 74(5): 563-78). The combination of THC and CBD in Sativex® is preferable to THC or CBD alone because THC is highly effective against neuropathic pain, and CBD counteracts possible side effects of THC, such as tachychardia, intoxication and sedation, while also contributing as an analgesic (Russo, Med Hypotheses. 2006; 66(2):234-46).

As indicated, THC and CBD are two examples of cannabinoids. Cannabinoids are a diverse class of compounds that interact with and activate cannabinoid receptors. There are three classes of cannabinoids: 1) endocannabinoids are naturally produced in the body by humans and other animals, 2) phytocannabinoids are produced by plants, and 3) synthetic cannabinoids are chemically produced cannabinoids. Synthetic cannabinoids can be identical to cannabinoids that are found in nature, or can be compounds that do not exist in nature.

Endocannabinoids are part of the endocannabinoid system including endogenous cannabinoids and cannabinoid receptors. Cannabinoid receptors (such as CB1 and CB2) are expressed in various cell types, including brain cells and immune cells. An example of an endocannabinoid is anandamide, a fatty acid neurotransmitter that interacts with cannabinoid receptors regulating the sensations of hunger, motivation, and pleasure.

Phytocannabinoids are produced by various types of plants, but the most well-known cannabinoid-producing plant is Cannabis. Cannabis plants produce over 60 cannabinoids, many of which, such as THC and CBD, have known therapeutic potential. Cannabis containing only trace amounts of THC is not psychoactive and is referred to as hemp. Cannabis species include Cannabis sativa, Cannabis indica, Cannabis ruderalis, and various hybrid indica/sativa crosses. In fact, hybrid crosses between Cannabis sativa and Cannabis indica are common.

Plants other than cannabis that produce cannabinoids include Echinacea purpurea, Echinacea angustifolia, Acmella oleracea, Helichrysum umbraculigerum, and Radula marginata. Cannabinoids isolated from Echinacea include lipophilic alkamides (alkylamides) such as cis/trans isomers dodeca-2E,4E,8Z,10E/Z-tetraenoic-acid isobutylamide.

Cannabis can have many medical uses including treatment of addiction (De Vries et al., Psychopharmacology (Berl). 2003 July; 168(1-2):164-9); ADHD (O'Connell and Che, Harm Reduction Journal. 2007; 4:16); alcoholism (Basavarajappa & Hungund, Alcohol. 2005 January-February; 40(1):15-24); Alzheimer's disease (Eubanks et al., Mol Pharm. 2006 November-December; 3(6):773-7); amyotrophic lateral sclerosis (ALS) (Raman et al., Amyotroph Lateral Scler Other Motor Neuron Disord. 2004 March; 5(1):33-9); anxiety (The British Journal of Psychiatry February 2001, 178 (2) 107-115); asthma (Tashkin et al., American Review of Respiratory Disease, 1975; 112, 377); auto-immune diseases (Lyman et al., J Neuroimmunol. 1989 June; 23(1):73-81); bacterial infections (Nissen et al., Fitoterapia. 2010 July; 81(5):413-9); bone loss (Bab et al., Ann Med. 2009; 41(8):560-7); brain injury/stroke (Shohami et al., Br J Pharmacol. 2011 August; 163(7):1402-10); cancer (Guindon & Hohmann, Br J Pharmacol. 2011 August; 163(7):1447-63); heart disease (Walsh et al., Br J Pharmacol. 2010 July; 160(5):1234-42); Huntington's disease (Lastres-Becker et al., J Neurochem. 2003 March; 84(5):1097-109); inflammation (AAPS J. 2009 March; 11(1): 109-119); Parkinson's disease (Sieradzan et al., Neurology. 2001 Dec. 11; 57(11):2108-11); and psoriasis (Trends Pharmacol Sci. 2009 August; 30(8): 411-420).

Additional documented uses for the cannabis plant include treating acquired hypothyroidism, acute gastritis, agoraphobia, ankyloses, arthritis, Asperger's syndrome, atherosclerosis, autism, bipolar disorder, blood disorders, cachexia, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, hepatitis, herpes, hypertension, impotence, incontinence, infant mortality, inflammatory bowel disease (IBD), insomnia, liver fibrosis, mad cow disease, menopause, migraine headaches, motion sickness, MRSA, muscular dystrophy, nail patella syndrome, neuroinflammation, nicotine addiction, obesity, obsessive compulsive disorder (OCD), pancreatitis, panic disorder, periodontal disease, phantom limb pain, poison ivy allergy, premenstrual syndrome (PMS), proximal myotonic myopathy, post-traumatic stress disorder (PTSD), Raynaud's disease, restless leg syndrome, schizophrenia, scleroderma, septic shock, shingles herpes zoster), sickle cell disease, seizures, sleep apnea, sleep disorders, stress, stuttering, temporomandibular joint disorder (TMJ), tension headaches, tinnitus, Tourette's syndrome, traumatic memories, wasting syndrome, and withdrawal.

While many phytochemicals present in cannabis are thought to contribute to the plant's medical and/or physiological benefits, THC and CBD have been the most extensively studied.

THC is the major psychoactive component of cannabis. THC exhibits complex effects on the central nervous system (CNS), including central sympathomimetic activity. THC's efficacy in pain treatment has been described in Pharm. J. 1997. 259, 104, and in Pharm. Sci. 1997. 3, 546. Oral THC is also useful in the treatment of AIDS symptoms, such as weight loss and pain (J. Pain. Symptom Manage. 1995. 10, 89-97). Furthermore, THC has anti-emetic properties and is used to control nausea and vomiting associated with cancer chemotherapy. THC also effects mood, cognition, memory and perception. These effects appear to be dose related.

CBD is another cannabinoid with a wide range of medicinal and/or physiological uses. CBD is antiemetic, neuroprotective, anti-inflammatory (Grotenhermen, et al., Int. 2012. 109(29-30), anxiolytic, anti-psychotic, and anti-arthritic (Burstein, Bioorgan Med Chem. 2015. 23, 1377-1385). Unlike THC, CBD does not exhibit psychoactive effects.

The physiological effects of THC and CBD are affected by the presence of other cannabis-derived molecules, such as additional cannabinoids, terpenes, and flavonoids. Terpenes are plant-derived molecules that often have distinctive potent aromas and can have desirable effects. For example, the terpene linalool is responsible for the aroma and relaxing properties of lavender. Flavonoids are a class of molecules that are ubiquitous among plants, and several are consumed as nutritional supplements for their antioxidant properties. Certain terpenes and flavonoids can interact with cannabinoid receptors, and this is thought to be one reason they contribute to the effects of cannabis. Consumption of THC and/or CBD in combination with other cannabis-derived molecules can enhance the desired effects of THC and/or CBD, and the combinatory action of cannabis-derived molecules can be referred to as entourage effects. Entourage effects may enhance the therapeutic potential of cannabinoids such as THC and CBD, with respect to pain, inflammation, depression, anxiety, addition, epilepsy, cancer, and infections (Russo, E. 2011. British Journal of Pharmacology. 163(7): 1344-1364). Entourage effects may also counteract THC side effects, such as dysphoria, and/or may enhance cannabis-induced euphoria.

Entourage effects also play a large role in the distinct effects of different cannabis strains. Entourage effects can contribute to the sedating, energizing, concentration-enhancing, relaxing, and/or other effects induced by particular cannabis strains.

Smoking is the most common route of cannabis consumption. When cannabis is heated for smoking, cannabinoid molecules become decarboxylated, to create the physiologically active form of the molecules (e.g., tetrahydrocannabivarinic acid or THCA becomes THC). However, smoking presents health hazards and can be irritating to the lungs and airways, so it is not an option for many cannabis users.

Cannabis is also often consumed orally, however this route of consumptions leads to slow onset of effect. Whereas smoking cannabis can lead to almost instant effects, oral administration provides an onset of action of 0.5 to 1 hour and a peak effect at 2-4 hours. The duration of action for psychoactive effects can be 4-6 hours, but the appetite stimulant effect may continue for 24 hours or longer after administration. In addition to slow onset, poor bioavailability of cannabinoids is another potential drawback to oral consumption of cannabis. However, due to a combined effect of first pass hepatic metabolism and low water solubility, only 10-20% of the administered dose reaches the systemic circulation. Therefore, oral consumption of cannabis is characterized by low bioavailability of cannabinoids, and slow onset of action.

Smoked cannabis undergoes thermal decarboxylation in situ during the smoking process. Cannabis for oral consumption however, must be decarboxylated prior to ingestion. This is generally accomplished by heating. Unfortunately, this heat processing of cannabis often leads to the loss of heat-labile molecules such as terpenes and flavonoids. Additionally, orally-consumed synthetic cannabinoids do not contain many phytochemicals that are present in whole cannabis. Therefore, orally consumed cannabis products and/or cannabinoids typically lack phytochemicals that contribute to entourage effects.

### SUMMARY OF THE DISCLOSURE

The current disclosure provides fast-acting oral formulations of plant-derived compositions with restored entourage effects. The formulations provide fast-acting delivery of the compositions by including an absorption enhancing carrier, such as an N-acylated fatty amino acid. Entourage effects are restored in the oral formulations of cannabis-derived molecules by including, with one or more primary cannabinoids, additional cannabinoids, terpenes, flavonoids, and/or other entourage-restoring molecules. Combining one or more primary cannabinoids (such as THC and/or CBD) with entourage-restoring molecules can restore and/or enhance certain physiological effects of cannabis, and the presence of an absorption enhancing carrier provides rapid and controlled delivery of the compositions.

In particular embodiments, carriers include N-acylated fatty amino acids, absorption enhancing agents, and/or various other beneficial carriers, such as surfactants, detergents, azones, pyrrolidones, glycols and bile salts. In particular embodiments, N-acylated fatty amino acids can be linear, branched, cyclic, bicyclic, or aromatic including, for example, 1-50 carbon atoms.

In particular embodiments, the one or more primary cannabinoids are cannabinoids that exert the major physiological effects of cannabis. In particular embodiments, the one or more primary cannabinoids include THC and/or CBD, or derivatives and/or analogs thereof.

In particular embodiments, the entourage-restoring molecules include additional cannabinoids. In particular embodiments, the additional cannabinoids can include Δ8-tetrahydrocannabinol (Δ8-THC), Δ11-tetrahydrocannabinol (Δ11-THC), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and/or tetrahydrocannabivarinic acid (THCVA).

In particular embodiments, the entourage-restoring molecules can include one or more terpenes. The one or more terpenes can include β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, sabinene and/or phellandrene.

In particular embodiments, the entourage-restoring molecules can include one or more flavonoids. The one or more flavonoids can include cannaflavin A, cannaflavin B, cannaflavin C, vitexin, isovitexin, apigenin, kaempferol, quercetin, luteolin, cinnamaldehyde, and/or orientin.

In particular embodiments, the entourage-restoring molecules can additionally include aroma and flavor conferring volatile compounds derived from cannabis. Aroma and flavor conferring molecules derived from cannabis include 2-heptanone, methyl heptanoate, methyl salicylate, methyl anthranilate, and hexanal.

The fast-acting oral formulations with restored entourage effects can create various administration benefits in a variety of conditions. Exemplary administration benefits include increased absorption, increased bioavailability, faster onset of action, higher peak concentrations, faster time to peak concentrations, increased subjective efficacy, increased objective efficacy, improved taste, and/or improved mouthfeel.

### BRIEF DESCRIPTION OF THE FIGURES

FIGS. 1A and 1B show an established correlation between water-solubility and the ability of SNAC to improve a molecule's absorption. FIG. 1A shows the multiple of improvement from SNAC plotted for cromolyn, vitamin B12, atorvastatin, and ibandronate, along with the aqueous solubility of each molecule. The plotted data shows a striking fit to a logarithmic trendline (R²=0.998), indicating a logarithmic relationship between the aqueous solubility of each and the extent to which SNAC improves absorption. As the water solubility of the molecule increases, SNAC's ability to enhance its absorption also increases. FIG. 1B plots the aqueous solubility of heparin, acyclovir, rhGH, PTH, MT-II, GLP-1, calcitonin, yy peptide, and THC according to the logarithmic trendline derived from FIG. 1A.

FIG. 2 provides modified amino acids of compounds I-XXXV.

FIG. 3 provides fatty acid amino acids of formulas (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CH3 (i.e. methyl group), and R3 is H; or a salt or the free acid form thereof.

FIG. 4 provides exemplary cannabinoid structures.

FIG. 5 provides various cannabis-derived molecules such as terpenes and flavonoids.

FIGS. 6A and 6B provide the average results of the study comparing onset and duration of action of orally administered cannabis/N-[8-(2-hydroxybenzoyl) amino] caprylate (SNAC) formulation and cannabis (without SNAC) formulation. FIG. 6A provides a bar graph of the results, with the SNAC formulation results depicted with black bars, and the results for the formulation without SNAC depicted with white bars. FIG. 6B provides a line graph of the results, with the SNAC formulation results depicted with circles, and the results for the formulation without SNAC depicted with triangles.

FIGS. 7A-7F provide the results for each individual participant in the study comparing onset and duration of action of orally administered cannabis/N-[8-(2-hydroxybenzoyl) amino] caprylate (SNAC) formulation (black bars) and cannabis (without SNAC) formulation white bars). FIG. 7A shows results for Study Participant No. 1 (“S1”); FIG. 7B shows results for Study Participant No. 2 (“S2”); FIG. 7C shows results for Study Participant No. 3 (“S3”); FIG. 7D shows results for Study Participant No. 4 (“S4”); FIG. 7E shows results for Study Participant No. 5 (“S5”) and FIG. 7F shows results for Study Participant No. 6 (“S6”).

FIG. 8 shows a comparison of intensity, duration and onset of action of orally administered cannabis formulations with a high SNAC dose (200 mg, “high dose”), a low SNAC dose (100 mg, “low dose”) and no SNAC (“control”).

FIG. 9 shows intensity, duration and onset of action of cannabis formulated with SNAC administered orally (“PO”) compared to cannabis administered by inhalation (“INH”).

FIG. 10 shows THC and CBD Cₘₐₓ and AUC following a single oral administration to rats.

FIG. 11 shows THC and CBD Cₘₐₓ (ng/ml) and AUC (hr*ng/mL) following a single oral administration to rats.

FIG. 12 shows intensity, duration and onset of action of orally administered cannabis/N-[8-(2-hydroxybenzoyl) amino] caprylic acid (NAC, “test”) formulation and cannabis only (without NAC, “control”) formulation.

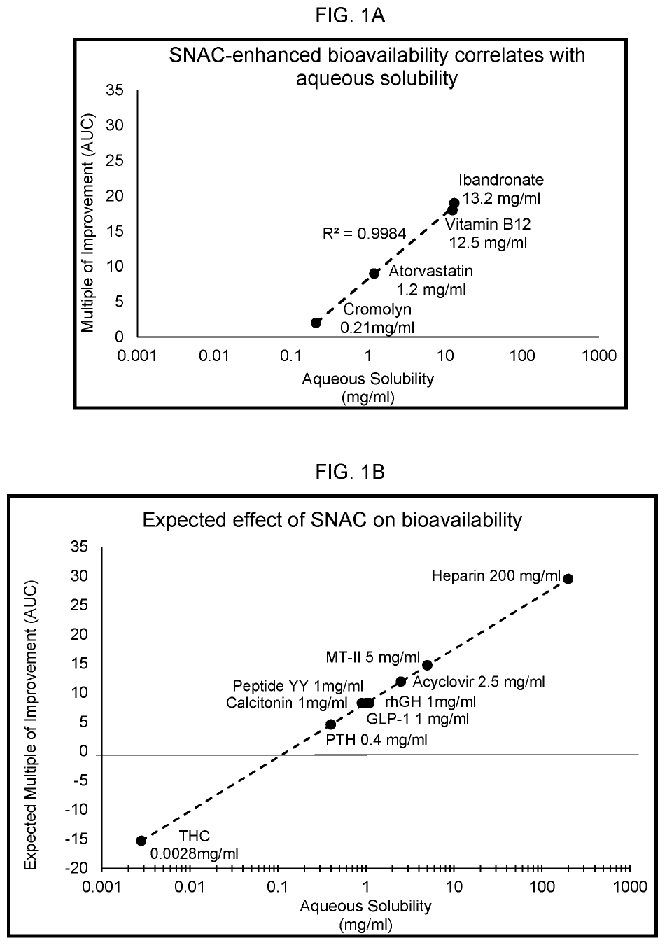

### DETAILED DESCRIPTION

The current disclosure provides fast-acting oral formulations of plant-derived compositions with restored entourage effects. The formulations provide fast-acting delivery of the compositions by including an absorption enhancing carrier, such as an N-acylated fatty amino acid. Entourage effects are restored in the oral formulation of cannabis-derived molecules by including, with one or more primary cannabinoids, additional cannabinoids, terpenes, flavonoids, and/or other entourage-restoring molecules. Combining one or more primary cannabinoids (such as THC and/or CBD) with entourage-restoring molecules can restore the physiological effects of certain cannabis extracts.

In particular embodiments, carriers include N-acylated fatty amino acids, absorption enhancing agents, and/or various other beneficial carriers, such as surfactants, detergents, azones, pyrrolidones, glycols and bile salts. In particular embodiments, N-acylated fatty amino acids can be linear, branched, cyclic, bicyclic, or aromatic including, for example, 1-50 carbon atoms.

In particular embodiments, the one or more primary cannabinoids include cannabinoids that exert the major physiological effects of cannabis. In particular embodiments, primary cannabinoids include THC and/or CBD, and/or derivatives and/or analogs thereof.

In particular embodiments, the fast-acting oral formulations include one or more entourage-restoring molecules. The one or more entourage-restoring molecules can include additional cannabinoids, terpenes, flavonoids, and/or aroma and flavor conferring volatiles.

In particular embodiments, the entourage-restoring molecules include one or more additional cannabinoids. In particular embodiments, the one or more additional cannabinoids can include Δ8-tetrahydrocannabinol (Δ8-THC), M1-tetrahydrocannabinol (Δ11-THC), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and/or tetrahydrocannabivarinic acid (THCVA).

In particular embodiments, the entourage-restoring molecules can include one or more terpenes. The one or more terpenes can include β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, sabinene and/or phellandrene.

In particular embodiments, the entourage-restoring molecules can include one or more flavonoids. The one or more flavonoids can include cannaflavin A, cannaflavin B, cannaflavin C, vitexin, isovitexin, apigenin, kaempferol, quercetin, luteolin, cinnamaldehyde, and/or orientin.

In particular embodiments, the entourage-restoring molecules can include one or more aroma and flavor conferring volatile compounds derived from cannabis. The one or more aroma and flavor conferring molecules derived from cannabis can include 2-heptanone, methyl heptanoate, methyl salicylate, methyl anthranilate, and/or hexanal.

The fast-acting oral formulations with restored entourage effects can create various administration benefits in a variety of conditions. Exemplary administration benefits include increased absorption, increased bioavailability, faster onset of action, higher peak concentrations, faster time to peak concentrations, increased subjective efficacy, increased objective efficacy, improved taste, and/or improved mouthfeel.

The following sections will describe in detail i) carriers that provide fast-acting delivery, ii) primary cannabinoids iii) molecules that restore entourage effects, iv) obtaining cannabis-derived molecules v) restoring entourage effects by combining cannabis-derived molecules, vi) formulating compositions with a carrier to provide fast-acting delivery, and vii) methods to provide physiological effects by delivering fast-acting formulations with restored entourage effects.

Carriers that Provide Fast-acting Delivery. Embodiments disclosed herein include one or more carriers that provide fast-acting delivery of components within the compositions. Fast-acting delivery can mean a faster onset of action of a composition, as compared to the onset of action of an equivalent composition lacking the carrier.

In particular embodiments, carriers disclosed herein create administration benefits selected from: increased absorption, increased bioavailability, faster onset of action, higher peak concentrations, faster time to peak concentrations increased subjective efficacy, increased objective efficacy, improved taste, and improved mouthfeel. Administration benefits related to increased absorption, increased bioavailability, faster onset of action, higher peak concentrations, and faster time to peak concentrations can alleviate adverse conditions more rapidly (for example, alleviation of pain). “Mouthfeel” refers to non-taste-related aspects of the pleasantness experienced by a person while ingesting (e.g., chewing or swallowing) an oral dosage form. Aspects of mouthfeel include the hardness and brittleness of a formulation, whether the formulation is chewy, gritty, oily, creamy, watery, sticky, easily dissolved, astringent, effervescent, and the like, and the size, shape, and form of the formulation (tablet, powder, gel, etc.). In particular embodiments, the administration benefit is a dose-dependent administration benefit. A dose-dependent administration benefit may refer to an administration benefit that occurs when the carrier is within a range of doses, or a range of relative doses (relative to an active ingredient). In particular embodiments, the dose-dependent administration benefits occur when the carrier is at a dose that is one to one hundred times or one to twenty times the dose of an active ingredient.

In particular embodiments, carriers include one or more modified amino acids, surfactants, detergents, azones, pyrrolidones, glycols, and bile salts.

An amino acid is any carboxylic acid having at least one free amine group and includes naturally occurring, non-naturally occurring and synthetic amino acids. Poly amino acids are either peptides or two or more amino acids linked by a bond formed by other groups which can be linked, e.g. an ester, anhydride, or an anhydride linkage. Peptides are two or more amino acids joined by a peptide bond. Peptides can vary in length from dipeptides with two amino acids to poly peptides with several hundred amino acids. See Chambers Biological Dictionary, editor: Walker, Cambridge, England: Chambers Cambridge, 1989, page 215. Di-peptides, tri-peptides, tetra-peptides, and penta-peptides can also be used.

Modified amino acid carriers include acylated fatty acid amino acids (FA-aa) or salts thereof, which are typically prepared by modifying the amino acid or an ester thereof by acylation or sulfonation. Acylated fatty acid amino acids include N-acylated FA-aa or an amino acid acylated at its alpha amino group with a fatty acid.

In particular embodiments, N-acylated fatty amino acids act as absorption enhancing agents, thereby creating an administration benefit. Absorption enhancing agents refer to compounds that promote gastrointestinal absorption. Absorption enhancing agents can improve drug absorption by improving the solubility of the drug in the gastrointestinal tract or by enhancing membrane penetration, as compared to a formulation that does not include the absorption enhancing agents. Additional examples of absorption enhancing agents include surfactants, detergents, azones, pyrrolidones, glycols or bile salts.

In particular embodiments, N-acylated fatty amino acids act as bioavailability enhancing agents. Bioavailability refers to the fraction of active ingredient that is actually absorbed by a subject and reaches the bloodstream. In particular embodiments, bioavailability enhancing agents increase the fraction of active ingredient in the bloodstream or result in detection of active ingredient in the bloodstream earlier in time, as compared to a formulation that does not include the bioavailability enhancing agent.

In particular embodiments, administration benefits created by absorption enhancing agents and/or bioavailability enhancing agents include faster onset of action, higher peak concentrations, faster time to peak concentrations, increased subjective efficacy, and/or increased objective efficacy as compared to a control formulation based on the same, similar in all aspects but for inclusion of the absorption enhancing agents and/or bioavailability enhancing agents.

Embodiments utilizing absorption enhancing agents and/or bioavailability enhancing agents (e.g., and in particular embodiments, N-acylated fatty amino acids) can be beneficial because many oral formulations designed to address various physiological conditions are characterized by a delayed onset of action and low bioavailability. These embodiments can allow more rapid absorption and higher bioavailability compared to formulations of cannabis-derived molecules ingested by currently available oral dosage forms.

Delayed onset of action presents challenges in indications that require rapid effect (e.g. pain and migraine); and low bioavailability requires patients to ingest significantly higher doses than would be required by alternative dosing forms (e.g. smoking, vaping). Particular embodiments disclosed herein provide oral formulations with improved bioavailability and shorter time to onset of effect.

As stated, in particular embodiments, N-acylated fatty amino acids act as subjective efficacy enhancing agents. Subjective efficacy enhancement refers to a noticeable physiological change, such as alleviation of a symptom, as perceived by a subject. In particular embodiments, subjective efficacy enhancing agents increase magnitude of a desired physiological effect, such as the alleviation of a symptom or induce the desired physiological effect more quickly, as compared to a formulation that does not include the subjective efficacy enhancing agent.

In particular embodiments, N-acylated fatty amino acids act as objective efficacy enhancing agents. Objective efficacy enhancement can refer to a physiological effect as determined by quantitative and/or qualitative measurement of an outcome. For example, objective efficacy enhancement can refer to alleviation of a clinical measure, such as a nutritional deficiency detected by a blood or saliva assay or a test of wellness, as administered by a physician. In particular embodiments, objective efficacy enhancing agents increase the alleviation of an objective clinical measure or result in alleviation more quickly, as compared to a formulation that does not include the objective efficacy enhancing agent.

Exemplary N-acylated fatty amino acid salts include sodium N-[8-(2-hydroxybenzoyl) amino] caprylate (SNAC). Other names for SNAC include Sodium-N-salicyloyl-8-aminocaprylate, Monosodium 8-(N-salicyloylamino) octanoate, N-(salicyloyl)-8-aminoactanoic add monosodium salt, monosodium N-{8-(2-hydroxybenzoyl)amino}octanoate, or sodium 84(2-hydroxybenzoyl)aminoioctanoate. SNAC has the structure:

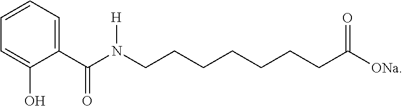

Salts of SNAC may also be used as a carrier.

Other forms of SNAC include:

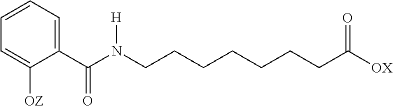

wherein X and Z are independently H, a monovalent cation, a divalent metal cation, or an organic cation. Examples of monovalent cations include sodium and potassium. Examples of divalent cations include calcium and magnesium. Examples of organic cations include ammonium and tetramethylammonium.

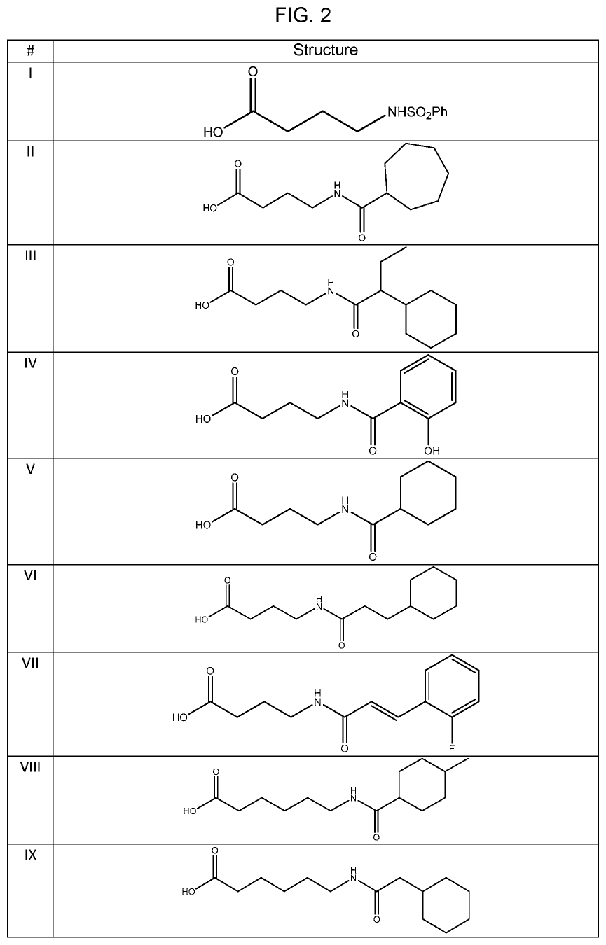

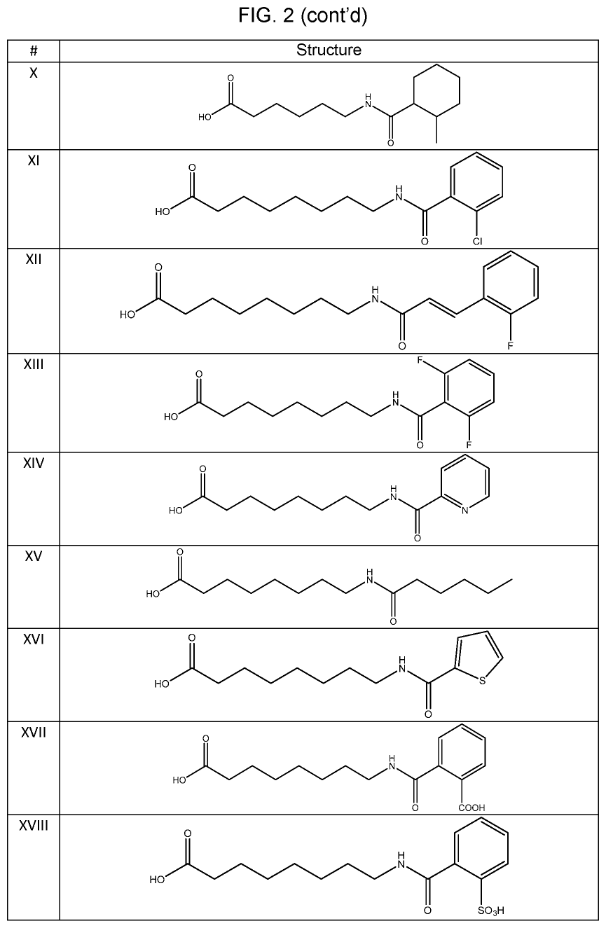

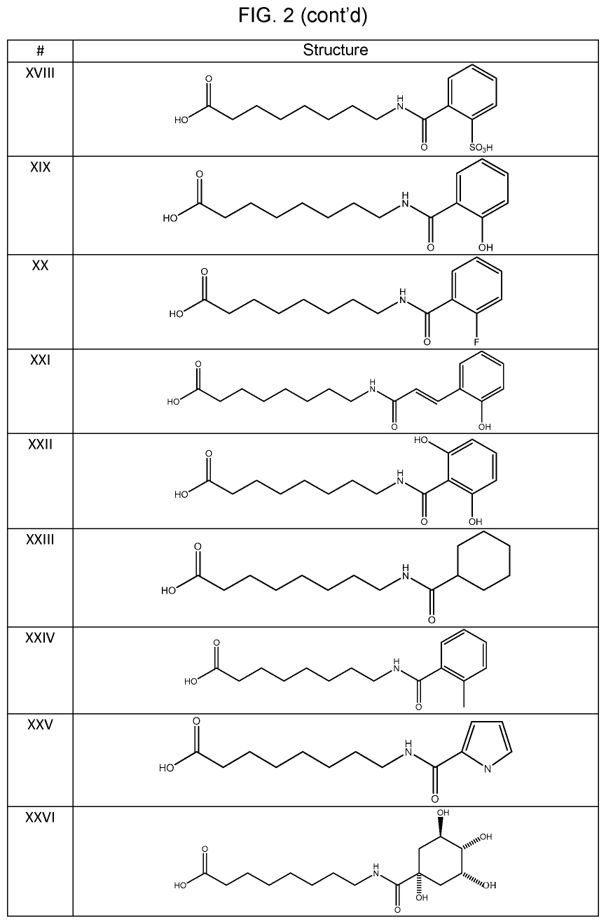

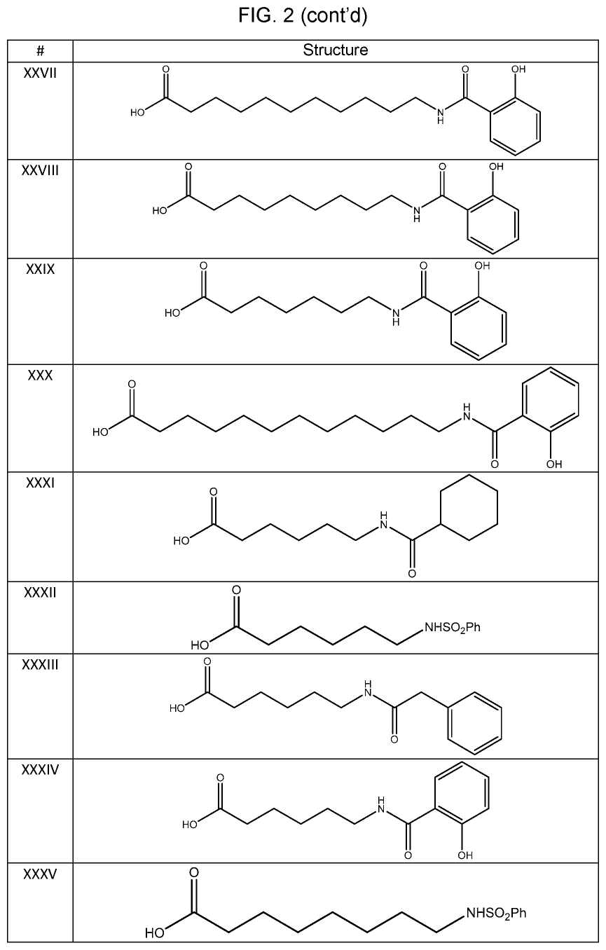

Exemplary modified amino acids, such as N-acylated FA-aas, are provided as compounds I-XXXV (see FIG. 2). Salts of these compounds and other N-acylated FA-aa can also be used as carriers.

Many of the compounds can be readily prepared from amino acids by methods within the skill of those in the art based upon the present disclosure. For example, compounds I-VII are derived from aminobutyric acid. Compounds VIII-X and XXXI-XXIIV are derived from aminocaproic acid. Compounds XI-XXVI and XXXV are derived from aminocaprylic acid. For example, the modified amino acid compounds above may be prepared by reacting the single amino acid with the appropriate modifying agent which reacts with free amino moiety present in the amino acids to form amides. Protecting groups may be used to avoid unwanted side reactions as would be known to those skilled in the art.

The amino acid can be dissolved in aqueous alkaline solution of a metal hydroxide, e.g., sodium or potassium hydroxide, and heated at a temperature ranging between 5° C. and 70° C., preferably between 10° C. and 40° C., for a period ranging between 1 hour and 4 hours, preferably 2.5 hours. The amount of alkali employed per equivalent of NH₂ groups in the amino acid generally ranges between 1.25 and 3 mmole, preferably between 1.5 and 2.25 mmole per equivalent of NH₂. The pH of the solution generally ranges between 8 and 13, preferably ranging between 10 and 12.

Thereafter, the appropriate amino acid modifying agent is added to the amino acid solution while stirring. The temperature of the mixture is maintained at a temperature generally ranging between 5° C. and 70° C., preferably between 10° C. and 40° C., for a period ranging between 1 and 4 hours. The amount of amino acid modifying agent employed in relation to the quantity of amino acid is based on the moles of total free NH₂ in the amino acid. In general, the amino acid modifying agent is employed in an amount ranging between 0.5 and 2.5 mole equivalents, preferably between 0.75 and 1.25 equivalents, per molar equivalent of total NH₂ group in the amino acid.

The reaction is quenched by adjusting the pH of the mixture with a suitable acid, e.g., concentrated hydrochloric acid, until the pH reaches between 2 and 3. The mixture separates on standing at room temperature to form a transparent upper layer and a white or off-white precipitate. The upper layer is discarded, and the modified amino acid is collected from the lower layer by filtration or decantation. The crude modified amino acid is then dissolved in water at a pH ranging between 9 and 13, preferably between 11 and 13. Insoluble materials are removed by filtration and the filtrate is dried in vacuo. The yield of modified amino acid generally ranges between 30 and 60%, and usually 45%.

If desired, amino acid esters, such as, for example benzyl, methyl, or ethyl esters of amino acid compounds, may be used to prepare the modified amino acids. The amino acid ester, dissolved in a suitable organic solvent such as dimethylformamide, pyridine, or tetrahydrofuran can be reacted with the appropriate amino acid modifying agent at a temperature ranging between 5° C. and 70° C., preferably 25° C., for a period ranging between 7 and 24 hours. The amount of amino acid modifying agent used relative to the amino acid ester is the same as described above for amino acids. This reaction may be carried out with or without a base such as, for example, triethylamine or diisopropylethylamine.

Thereafter, the reaction solvent is removed under negative pressure and the ester functionality is removed by hydrolyzing the modified amino acid ester with a suitable alkaline solution, e.g. 1N sodium hydroxide, at a temperature ranging between 50° C. and 80° C., preferably 70° C., for a period of time sufficient to hydrolyze off the ester group and form the modified amino acid having a free carboxyl group. The hydrolysis mixture is then cooled to room temperature and acidified, e.g. aqueous 25% hydrochloric acid solution, to a pH ranging between 2 and 2.5. The modified amino acid precipitates out of solution and is recovered by conventional means such as filtration or decantation. Benzyl esters may be removed by hydrogenation in an organic solvent using a transition metal catalyst.

The modified amino acid may be purified by recrystallization or by fractionation on solid column supports. Suitable recrystallization solvent systems include acetonitrile, methanol and tetrahydrofuran. Fractionation may be performed on a suitable solid column supports such as alumina, using methanol/n-propanol mixtures as the mobile phase; reverse phase column supports using trifluoroacetic acid/acetonitrile mixtures as the mobile phase; and ion exchange chromatography using water as the mobile phase. When anion exchange chromatography is performed, preferably a subsequent 0-500 mM sodium chloride gradient is employed.

In particular embodiments, modified amino acids having the formula

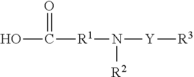

wherein Y is

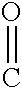

### or SO₂;

R¹ is C₃-C₂₄ alkylene, C₂-C₂₀ alkenylene, C₂-C₂₀ alkynylene, cycloalkylene, or an aromatic, such as arylene;
R² is hydrogen, C₁-C₄ alkyl, or C₂-C₄ alkenyl; and
R³ is C₁-C₇ alkyl, C₃-C₁₀ cycloalkyl, aryl, thienyl, pyrrolo, or pyridyl, and
R³ is optionally substituted by one or more C₁-C₅ alkyl group, C₂-C₄ alkenyl group, F, Cl, OH, OR¹, SO₂, COOH, COOR¹ or, SO₃H;
may be prepared by
reacting in water and the presence of a base a lactam having the formula

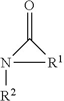

with a compound having the formula R³—Y—X, wherein Y, R¹, R², and R³ are as above and X is a leaving group. A lactam as shown in the above formula can be prepared, for example by the method described in Olah et al., Synthesis, 537-538 (1979).

In particular embodiments, modified amino acids also include an amino acid acylated at its alpha amino group with a fatty acid, which can be represented by the general formula A-X, wherein A is the alpha-amino acid residue and X is a fatty acid attached by acylation to A's alpha-amino group. The amino acids include cationic and non-cationic amino acids. In particular embodiments the term “non-cationic amino acid” refers to an amino acid selected from the group consisting of non- polar hydrophobic amino acids, polar non-charged amino acids, and polar acidic amino acids. In particular embodiments the term “non-cationic amino acid” as used herein refers to amino acids selected from the group consisting of Alanine (Ala), Valine (Val), Leucine (Leu), Isoleucine (Ile), Phenylalanine (Phe), Tryptophan (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gin), Aspartic acid (Asp), and Glutamic acid (Glu).

In particular embodiments, the acylated FA-aa includes an alpha amino acid residue of a non-polar hydrophobic amino acid. In particular embodiments, the acylated FA-aa may be represented by the general formula A-X, wherein A is the amino acid residue of a non-polar hydrophobic amino acid and X is a fatty acid attached by acylation to A's alpha-amino group. In particular embodiments the term “non-polar hydrophobic amino acid” as used herein refers to categorization of amino acids used by the person skilled in the art. In particular embodiments the term “non-polar hydrophobic amino acid” refers to an amino acid selected from the group consisting of Ala, Val, Leu, Ile, Phe, Trp, Met, Pro, and Sarcosine.

In particular embodiments, the acylated FA-aa includes the amino acid residue of a polar non-charged amino acid. In particular embodiments the acylated FA-aa may be represented by the general formula A-X, wherein A is the amino acid residue of a polar non-charged amino acid and X is a fatty acid attached by acylation to A's alpha-amino group. In particular embodiments the term “polar non-charged amino acid” as used herein refers to categorization of amino acids used by the person skilled in the art. In particular embodiments the term “polar non-charged amino acid” refers to an amino acid selected from the group consisting of Gly, Ser, Thr, Cys, Tyr, Asn, and Gin.

In particular embodiments, the acylated FA-aa includes the amino acid residue of a polar acidic amino acid. In particular embodiments, the acylated FA-aa may be represented by the general formula A-X, wherein A is the amino acid residue of a polar acidic amino acid and X is a fatty acid attached by acylation to A's alpha-amino group. In particular embodiments, the term “polar acidic amino acid” as used herein refers to categorization of amino acids used by the person skilled in the art. In particular embodiments, the term “polar acidic amino acid” refers to an amino acid selected from the group consisting of Asp and Glu.

In particular embodiments, the amino acid residue of the acylated FA-aa includes the amino acid residue of an amino acid that is not encoded by the genetic code. Modifications of amino acids by acylation may be readily performed using acylation agents known in the art that react with the free alpha-amino group of the amino acid.

In particular embodiments, the alpha-amino acids or the alpha-amino acid residues herein are in the L-form unless otherwise stated.

In particular embodiments, the amino acid residue is in the free acid form and/or a salt thereof, such as a sodium (Na+) salt thereof.

Exemplary embodiments of acylated FA-aas may be represented by the general Fa-aa formula I:

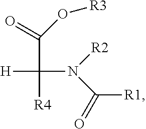

wherein R1 is an alkyl or aryl group including 5 to 19 carbon atoms; R2 is H (i.e. hydrogen), CH₃ (i.e. methyl group), or covalently attached to R4 via a (CH₂)₃ group; R3 is H or absent; and R4 is an amino acid side chain or covalently attached to R2 via a (CH₂)₃ group; or a salt thereof.

The FA-aa can be acylated with a fatty acid including a substituted or unsubstituted alkyl group consisting of 5 to 19 carbon atoms. In particular embodiments, the alkyl group consists of 5 to 17 carbon atoms. In particular embodiments, the alkyl group consists of 5-15 carbon atoms. In particular embodiments the alkyl group consists of 5-13 carbon atoms. In particular embodiments the alkyl group consists of 6 carbon atoms.

In particular embodiments, the acylated FA-aa is soluble at intestinal pH values, particularly in the range pH 5.5 to 8.0, such as in the range pH 6.5 to 7.0. In particular embodiments, the acylated FA-aa is soluble below pH 9.0.

In particular embodiments, the acylated FA-aa has a solubility of at least 5 mg/mL. In particular embodiments, the acylated FA-aa has a solubility of at least 10 mg/mL. In particular embodiments, the acylated FA-aa has a solubility of at least 20 mg/mL. In particular embodiments, the acylated FA-aa has a solubility of at least 30 mg/mL. In particular embodiments, the acylated FA-aa has a solubility of at least 40 mg/mL. In particular embodiments, the acylated FA-aa has a solubility of at least 50 mg/mL. In particular embodiments, the acylated FA-aa has a solubility of at least 60 mg/mL. In particular embodiments, the acylated FA-aa has a solubility of at least 70 mg/mL. In particular embodiments, the acylated FA-aa has a solubility of at least 80 mg/mL. In particular embodiments, the acylated FA-aa has a solubility of at least 90 mg/mL. In particular embodiments, the acylated FA-aa has a solubility of at least 100 mg/mL. In particular embodiments, solubility of the acylated FA-aa is determined in an aqueous solution at a pH value 1 unit above or below pKa of the FA-aa at 37° C. In particular embodiments, solubility of the acylated FA-aa is determined in an aqueous solution at pH 8 at 37° C. In particular embodiments, solubility of the acylated FA-aa is determined in an aqueous solution at a pH value 1 unit above or below pl of the FA-aa at 37° C. In particular embodiments, solubility of the acylated FA-aa is determined in an aqueous solution at a pH value 1 units above or below pl of the FA-aa at 37° C., wherein said FA-aa two or more ionisable groups with opposite charges. In particular embodiments, solubility of the FA-aa is determined in an aqueous 50 mM sodium phosphate buffer, pH 8.0 at 37° C.

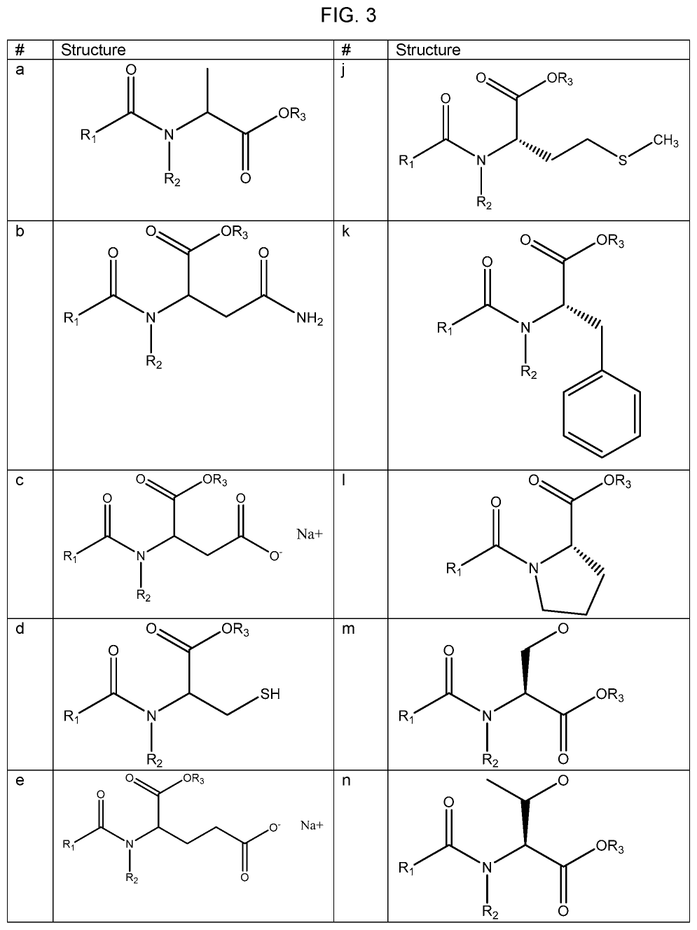

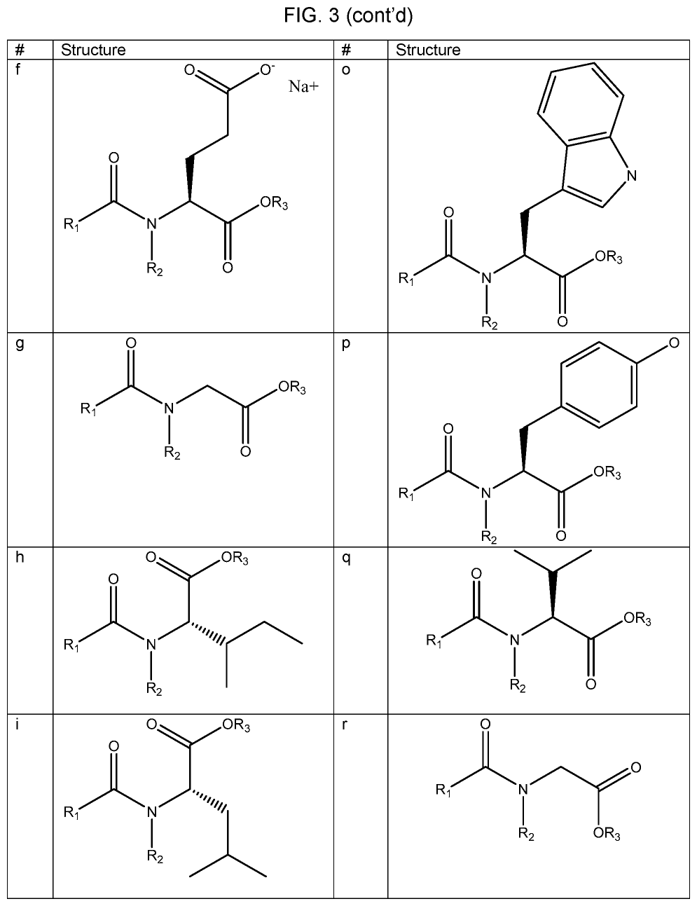

In particular embodiments the acylated FA-aa is selected from the group consisting of formula (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CHs (i.e. methyl group), and R3 is H; or a salt or the free acid form thereof. Formulas (a), (b), (c), (d), (e), (f), (g), (h), (i), (j), (k), (l), (m), (n), (o), (p), (q), and (r) are provided in FIG. 3.

In particular embodiments, the acylated FA-aa can be selected from one or more of sodium N-dodecanoyl alaninate, N-dodecanoyl-L-alanine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl tryptophanate, N-dodecanoyl-L-tryptophan, sodium N-dodecanoyl valinate, N-dodecanoyl-L-valine, sodium N-dodecanoyl sarcosinate, N-dodecanoyl-L-sarcosine, sodium N-oleoyl sarcosinate, sodium N-decyl leucine, sodium N-decanoyl alaninate, N-decanoyl-L-alanine, sodium N-decanoyl leucinate, N-decanoyl-L-leucine, sodium N-decanoyl phenylalaninate, N-decanoyl-L-phenylalanine, sodium N-decanoyl valinate, N-decanoyl-L-valine, sodium N-decanoyl isoleucinate, N-decanoyl-L-isoleucine, sodium N-decanoyl methioninate, N-decanoyl-L-methionine, sodium N-decanoyl prolinate, N-decanoyl-L-proline, sodium N-decanoyl threoninate, N-decanoyl-L-threonine, sodium N-decanoyl tryptophanate, N-decanoyl-L-tryptophan, sodium N-decanoyl sarcosinate, N-decanoyl-L-Sarcosine, N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl tyrosinate, N-decanoyl-L-tyrosine, sodium N-dodecanoyl asparaginate, sodium N-dodecanoyl glutamic acid, N-dodecanoyl-L-glutamic acid, sodium N-decanoyl glutamic acid, N-decanoyl-L-glutamic acid, Amisoft HS-11 P (sodium Stearoyl Glutamate, Amisoft MS-11 (sodium Myristoyl Glutamate), Amisoft LS-11 (sodium Dodecanoyl Glutamate), Amisoft CS-11 (sodium Cocoyl Glutamate), sodium N-cocoyl glutamate, Amisoft HS-11 P, Amisoft HS-11 P (sodium N-stearoyl glutamate), (sodium N-myristoyl glutamate)), (sodium N-dodecanoyl glutamate), and Amisoft HS-11 P.

The following acylated FA-aas are commercially available:

| | | Provider (per |
|---|---|---|
| Brand Name | Chemical Name | 14-APR-2011) |
| Hamposyl L-95 | sodium N-dodecanoyl sarcosinate | Chattem |
| | | Chemicals |
| Hamposyl O | sodium N-oleoyl sarcosinate | Chattem |
| | | Chemicals |
| Hamposyl C | sodium N-cocoyl sarcosinate | Chattem |
| | | Chemicals |
| Hamposyl L-30 | sodium N-dodecanoyl sarcosinate | Chattem |
| | | Chemicals |
| Amisoft HS-11 P | sodium N-stearoyl glutamate | Ajinomoto |
| Amisoft LS-11 | sodium N-dodecanoyl glutamate | Ajinomoto |
| Amisoft CS-11 | sodium N-cocoyl glutamate | Ajinomoto |
| Amisoft MS-11 | sodium N-myristoyl glutamate | Ajinomoto |
| Amilite GCS-11 | sodium N-cocoyl glycinate | Ajinomoto |

In particular embodiments the terms “fatty acid N-acylated amino acid”, “fatty acid acylated amino acid”, or “acylated amino acid” are used interchangeably herein and refer to an amino acid that is acylated with a fatty acid at its alpha-amino group.

Primary cannabinoids. The fast-acting oral formulations also include one or more primary cannabinoids. In particular embodiments, primary cannabinoids are cannabinoids that exert the primary desired physiological effects of cannabis. Examples of cannabinoids that exert primary desired physiological effects of cannabis include Δ9-tetrahydrocannabinol (THC) and cannabidiol (CBD). Primary cannabinoids can also include derivatives and/or analogs of a cannabinoid that exert a primary desired physiological effect of cannabis.

The term “derivative” refers to a compound that is obtained from a similar compound or a precursor compound by a chemical reaction. The term “analog” (also “structural analog” or “chemical analog”) refers to a compound that is structurally similar to another compound but differs with respect to certain components, such as an atom, a functional group, and/or a substructure. Examples of analogs of THC include nabilone, ajulemic acid, and (−) HU-210. An example of a CBD analog is abn-CBD.

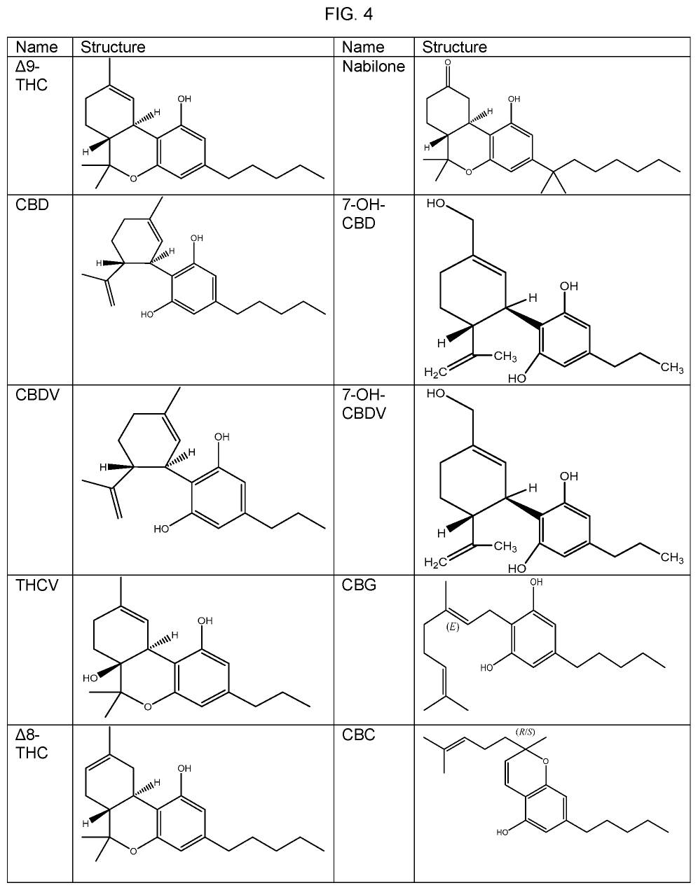

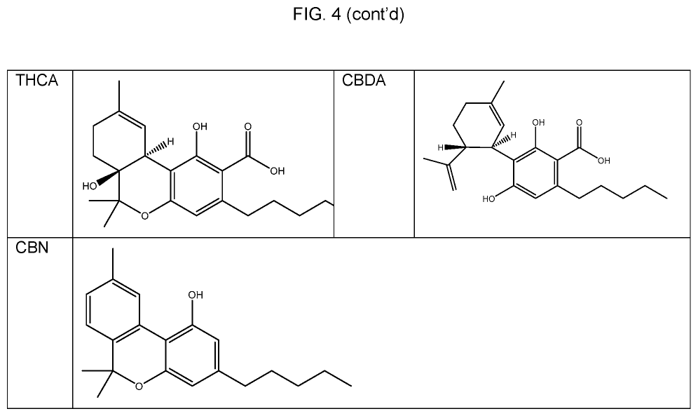

In particular embodiments, the primary cannabinoids include THC. THC is the predominant cannabinoid present in many cannabis strains, at often 10-20% of the dry weight of cannabis flowers. THC content in cannabis can vary from trace amounts (<1%) to over 30%. Many THC dominant cannabis strains contain only trace amounts of CBD (<1%). An example of a THC-dominant, low-CBD cannabis strain is Sour Diesel (22% THC and 0.1% CBD). Cannabis and/or cannabis extracts containing THC (>1%) can be useful to provide a physiological and/or medical benefit of THC. An exemplary structure of THC is shown in FIG. 4.

In particular embodiments, the primary cannabinoids include CBD. In certain cannabis strains, such as Charlotte's Web™ (Stanley Brothers Social Enterprises, LLC, Colorado Springs, Colo.), CBD is the predominant cannabinoid. Charlotte's WebTM contains an average of 20% CBD and trace amounts of THC (0.3%), as measured by dry weight in cannabis flowers. CBD-dominant (>1%), low-THC (<1%) cannabis strains can be used for medicinal and nutritional benefits, and can be desirable in certain situations because they lack the psychoactive effects of THC. CBD content in cannabis can range from trace amounts (<1%) to over 20%. An exemplary structure of CBD is shown in FIG. 4.

In particular embodiments, the fast-acting oral formulations include a combination of THC and CBD. Examples of cannabis strains that contain THC and CBD (over 1% each) include Harlequin, (5% THC and 12% CBD) and CBD Mango Haze (14% THC and 16% CBD). The health benefits of THC and CBD can be enhanced when the two molecules are provided together. For example, a combination of THC and CBD is thought to optimize certain analgesic and anxiolytic properties of the two cannabinoids. Furthermore, CBD can reduce or eliminate negative side effects of THC. The ratio of THC:CBD in cannabis strains can range from >100:1 THC:CBD to <0.01:1 THC:CBD.

In particular embodiments, the primary cannabinoids include nabilone. Nabilone is a synthetic THC analog that is used for anxiolytic and antiemetic properties, and is also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7). Nabilone is commonly administered in 1-2 mg doses, up to 6 mg per day. An exemplary structure of nabilone is shown in FIG. 4.

Molecules that Restore Entourage Effects. In particular embodiments, the fast-acting oral formulations include one or more entourage-restoring molecules. Entourage-restoring molecules can refer to molecules that, when provided in a composition with THC and/or CBD, restore or enhance particular desired effects, as compared to the effects of THC and/or CBD alone. In particular embodiments the one or more entourage-restoring molecules can include additional cannabinoids, terpenes, flavonoids, and/or aroma and flavor conferring volatiles.

Additional cannabinoids. In particular embodiments the entourage-restoring molecules include additional cannabinoids (in addition to the primary cannabinoids THC and/or CBD). Cannabis produces over 60 different cannabinoids (Brenneisen, Marijuana and the Cannabinoids, Ch. 2, 2007, Humana Press). Cannabinoids are made by the trichome secretory glands of cannabis, which are highly concentrated in the flowers of female plants. Cannabinoids can also be found in other parts of the cannabis plant, including the stems and leaves.

Cannabinoids other than THC and CBD contribute to the various physiological effects of cannabis. For example, cannabigerol (CBG) counteracts THC-induced paranoia and is anti-inflammatory, anti-bacterial, and anxiolytic. Cannabichromene (CBC) is anti-inflammatory and analgesic. Cannabinol (CBN), which is produced by the degradation of THC, is analgesic, anxiolytic, and has mildly psychoactive effects. Tetrahydrocannabivarin (THCV), in contrast to THC, is an appetite suppressant.

Cannabinoid content can vary widely depending on plant strain, age, growth conditions, and storage conditions. Cannabis strains vary in their non-THC, non-CBD cannabinoid content. For example, whereas the strain Purple Kush contains 0.02% CBN; 0.4% CBG; 0.1% THCV; 0.05% CBC; and 0.1% CBL, the strain Durban Poison contains 0.1% CBN; 1% CBG; 1% THCV; 0.05% CBC; and 1.2% CBL (averages reported by Steep Hill Labs, Inc.). The variation in cannabinoid content across cannabis strains contributes to the distinct entourage effects of each strain.

In particular embodiments, the entourage-restoring molecules include one or more of Δ8-tetrahydrocannabinol (Δ8-THC), Δ11-tetrahydrocannabinol (Δ11-THC), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA).

Terpenes. In particular embodiments, the entourage-restoring molecules include terpenes. Terpenes can refer to terpenes or terpenoids, or derivatives and/or analogs thereof. Terpenes are a large class of organic molecules that include one or more units of isoprene (C₅H₈). Terpene molecules that include additional functional groups are also known as terpenoids. The isoprene units of terpenes can be linked together to form linear molecules or rings. Terpenes can be classified by the number of isoprene subunits present. For example, hemiterpenes contain one isoprene subunit, monoterpenes contain two isoprene subunits, sesquiterpenes contain three isoprene subunits, and diterpenes contain four isoprene subunits.

In particular embodiments, the entourage-restoring molecules include one or more cannabis-derived terpenes. More than 100 terpenes have been identified in cannabis plants (Rothschild et al., Bot J Linn Soc. 2005. 147(4):387-397 and Brenneisen “Forensic Science and Medicine: Marijuana and the Cannabinoids” Chapter 2, ed. M ElSohly, Humana Press New York, N.Y., 2007). Like cannabinoids, terpenes are produced by cannabis trichome glands, which are concentrated in cannabis flowers. However, terpenes can also be found in other part of the cannabis plant, such as in stems and leaves. Examples of cannabis-derived terpenes include β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, and phellandrene. In particular embodiments, the one or more terpenes include alloaromadendrene, (Z)-α-cis-bergamotene, (Z)-α-trans-bergamotene, β-bisabolol, epi-α-bisabolol, β-bisabolene, borneol (camphol), cis-γ-bisabolene, borneol acetate (bornyl acetate), α-cadinene, cis-carveol, α-humulene (α-caryophyllene), γ-cadinene, Δ-3-carene, caryophyllene oxide, 1,8-cineole, citral A, citral B, α-copaene (aglaiene), γ-curcumene, p-cymene, β-elemene, γ-elemene, eucalyptol, α-eudesmol, β-eudesmol, γ-eudesmol, eugenol, cis-β-farnesene (O-β-farnesene), trans-α-farnesene, trans-β-farnesene, trans-γbisabolene, fenchone, fenchol (norbornanol, β-fenchol), α-guaiene, ipsdienol, lemenol, d-limonene, linalyl alcohol (β-linolool), α-longipinene, menthol, γ-muurolene, trans-nerolidol, nerol, β-ocimene (cis-ocimene), α-phellandrene, 2-pinene, sabinene, cis-sabinene hydrate (cis-thujanol), β-selinene, α-selinene, γ-terpinene, isoterpine, terpineol (a-terpineol), terpineol-4-ol, α-terpinene (terpinene), α-thujene (origanene), viridiflorene (ledene), and/or α-ylange.

In particular embodiments, the terpenes include linalool. Linalool is a monoterpene that naturally occurs in many plants including cannabis, lavender, bay laurel, citrus fruits, and mint, among others. Linalool naturally exists as two isomers, known as licareol and coriandrol. Multiple studies have demonstrated anti-inflammatory (Peana et al., Phytomedicine 2002. 9(8):721-6), analgesic (Peana et al., Eur J Pharmacol 2003 460(1):37-41) and anti-anxiety (Linck et al., Phytomedicine 2002. 17(8-9):679-83; Souto-Major et al., Pharmacol Biochem Behav 2011. 100(2):259-63) effects of linalool. Linalool is commonly used as a food additive and is Generally Recognized as Safe by the U.S. Food and Drug Administration (FDA).

In particular embodiments, the terpenes include nerolidol. Nerolidol is a cannabis-derived terpene that has sedative properties (Binet et al., Ann Pharm Fr 1972. 30:611-616). Therefore, nerolidol contributes to the sedative effects of particular cannabis strains.

In particular embodiments, the terpenes include pinene. Pinene is a monoterpene that exists as two isomers, α-pinene and β-pinene. Pinene has a pine-like scent and naturally occurs in pine trees and cannabis. Pinene has anti-inflammatory effects (Gil et al., Pharmazie 1989. 44(4):284-7), anti-microbial properties (Nissen et al., Fitoterapia 2010. 81(5):413-19), and is a bronchodilator at low concentration (Falk et al., Scand J Work Environ Health 1990. 16:372-378). Pinene may also improve memory (Perry, et al, Journal of Pharmacy and Pharmacology 2000. 52(7):895-902), and therefore is thought to counteract the short-term memory impairment that can be induced by THC.

In particular embodiments, the terpenes include caryophyllene (or β-caryophyllene). β-caryophyllene is a sesquiterpene that naturally occurs in rosemary, hops, cannabis, cloves, black pepper, lavender, caraway, basil, and cinnamon. B-caryophyllene has anti-inflammatory (Gertsch et al., PNAS. 2008. 105(26):9099104), anti-analgesic (Katsuyama et al., European Journal of Pain. 2013. 17(5): 664-675), neuroprotective (Guimaraes-Santos, J Evid Based Complementary Altern Med. 2012. 1-9), anti-anxiety and anti-depressant (Bahi et al., Physiology & Behavior. 2014. 135:119-124) effects. Caryophyllene is a direct agonist of the cannabinoid receptor CB2, which is present on immune cells, and can enhance the anti-inflammatory properties of cannabis.

In particular embodiments, the terpenes include limonene. Limonene is a monoterpene that naturally occurs in citrus trees, citronella grass, verbena plants, and cannabis. Limonene is the main component of citrus fruit that confers the citrus-like aroma. Limonene has anti-inflammatory (Piccinelli et al. Life Sci. 2016 S0024-3205(16):30669-5) and anti-depressant effects (Komori et al., 1995). Limonene is commonly used as a food additive and is Generally Recognized as Safe by the U.S. F.D.A.

In particular embodiments, the terpenes include β-myrcene. β-myrcene is a monoterpene that is commonly found in hops, parsley, thyme, bay leaves, mangoes, lemongrass and cannabis. β-myrcene has analgesic (Paula-Freire et al., Planta Med. 2016; 82(3):211-6), anti-inflammatory (Lorenzetti et al., J of Ethnopharmacology. 1991.34(1):43-48), anti-microbial (Yoshihiro et al., Natural Medicines. 2004. 58(1), 10-14), and sedative (Rao et al., J Pharm Pharmacol. 1990. 42(12): 877-878) effects. Indica strains of cannabis are characteristically high in β-myrcene (>0.5%), and β-myrcene contributes to the sedating, “couch-lock” inducing effects of indica or indica-dominant strains.

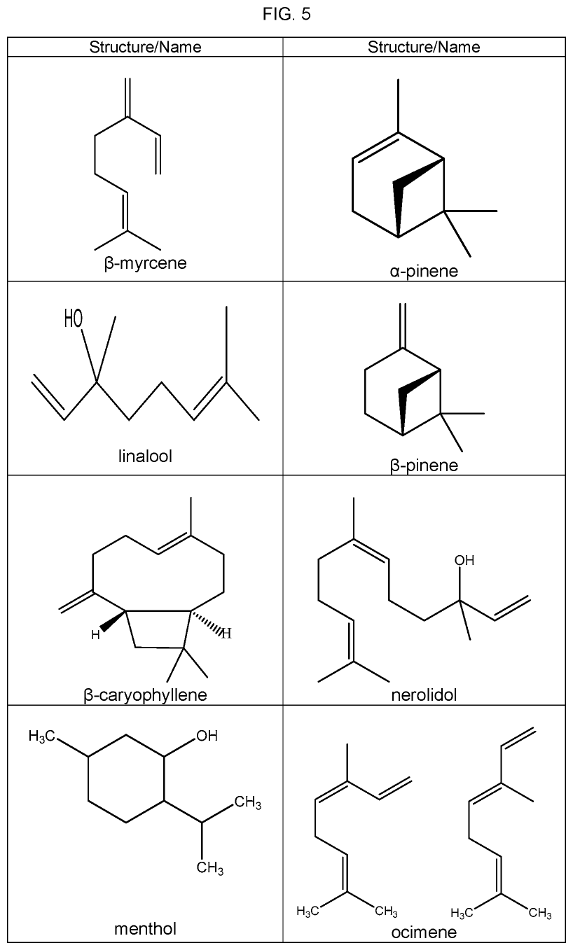

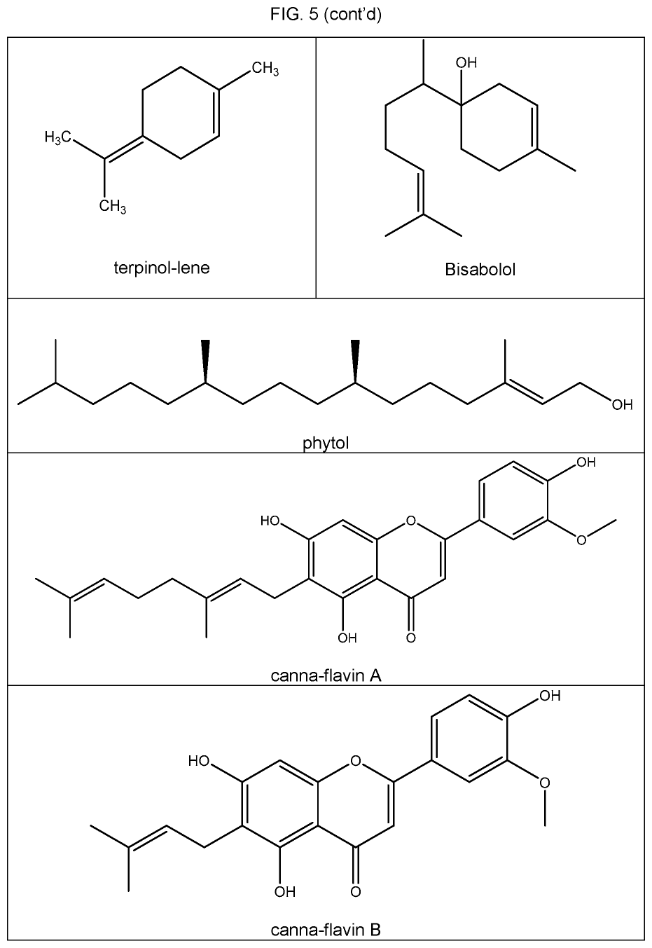

Flavonoids. In particular embodiments the entourage-restoring molecules include flavonoids. Flavonoids are a class of secondary metabolite found in plants and fungus that each contain a 15- carbon skeleton including two phenyl rings and a heterocyclic ring. Flavonoids can be classified into three groups: i) bioflavonoids or flavonoids, ii) isoflavonoids, and iii) neoflavonoids. Flavonoids that naturally occur in cannabis plants include cannaflavin A, cannaflavin B, cannaflavin C, vitexin, isovitexin, apigenin, kaempferol, quercetin, luteolin, cinnamaldehyde, and orientin. See FIG. 5 for exemplary structures of particular flavonoids.

In particular embodiments, the entourage-restoring molecules include cannaflavin A, cannaflavin B, and/or cannaflavin C. Cannaflavins are flavonoids that are unique to cannabis plants. Cannaflavin A and cannaflavin B both have anti-inflammatory activity (Barrett et al., Experientia 1986. 15;42(4):452-3).

In particular embodiments, the entourage-restoring molecules include apigenin. Apigenin is a flavonoid that naturally occurs in many plants, such as cannabis, parsley, celery and chamomile. Apigenin is an opioid receptor agonist and has many beneficial health effects, including specifically inducing death of cancer cells, anxiolytic activity (Salgueiro et al., Pharmacol Biochem Behav 1997. 58, 887-891) and stimulation of neurogenesis.

In particular embodiments, the entourage-restoring molecules include kaempferol. Kaempferol is a flavonoid commonly found in many plant-based foods, including apples, grapes, tomatoes, potatoes, onions, broccoli, squash, cucumber, and berries. There are a wide range of positive health effects of ingesting kaempferol. For example, kaempferol has antioxidant, anti-inflammatory, antimicrobial, anti-cancer, cardioprotective, neuroprotective, antidiabetic, anti-osteoporotic, anxiolytic, analgesic and antiallergic properties (Calderon-Montano et al., Mini Rev Med Chem. 2011. 11(4):298-344).

In particular embodiments, the entourage-restoring molecules include quercetin. Quercetin is a flavonoid found in many plants, including cannabis, kidney beans, capers, cilantro, onion, kale, plum, cranberry and sweet potato. Quercetin may have antioxidant and anti-cancer effects (Alam et al., Environ Sci Pollut Res Int 2016).

In particular embodiments, the entourage-restoring molecules include orientin. Orientin is a flavonoid that can be found in cannabis, passion flower, Acai palm, barley and millet. Medicinal properties of orientin include antioxidant, antiaging, antimicrobial, antiinflammatory, vasodilatation, radiation protective, neuroprotective, antidepressant, anti-adipogenesis, and antinociceptive effects (Lam et al., Adv Pharmacol Sci. 2016. 2016:4104595).

Other cannabis-derived molecules. In particular embodiments the entourage-restoring molecules include other cannabis-derived molecules. In addition to terpenes and flavonoids, certain volatile compounds confer distinct aroma and flavor profiles to cannabis. Examples of aroma and flavor conferring volatiles that are present in cannabis are listed in Rice & Koziel. PLoS One. 2015. 10(12):e0144160. Particular cannabis-derived molecules that contribute to cannabis aroma and flavor include 2-heptanone, methyl heptanoate, methyl salicylate, methyl anthranilate, and hexanal. These molecules are volatile compounds, meaning that they have a high tendency to vaporize. Therefore, aroma and flavor conferring volatile compounds of cannabis are often lost during production of cannabis extracts for human consumption. The molecules 2-heptanone, methyl heptanoate, methyl salicylate, methyl anthranilate, and hexanal, as well as other volatile cannabis-derived compounds, are U.S. F.D.A. approved food additives. In particular embodiments, low concentrations (1% or lower) of these volatiles can be used to confer particular aromas and flavors to oral formulations described herein.

Obtaining Cannabis-Derived Molecules. In particular embodiments the fast-acting oral formulations with restored entourage effects include one or more primary cannabinoids (THC and/or CBD) and one or more entourage-restoring molecules. Extraction and decarboxylation of THC and/or CBD for oral consumption can lead to loss of entourage effect molecules. Therefore, compositions with THC and/or CBD can be supplemented with cannabis-derived molecules to restore entourage effects.

Primary Cannabinoids. In particular embodiments, decarboxylated cannabis extracts are included in the formulations to provide primary cannabinoids. Decarboxylated cannabis extracts containing THC and/or CBD can be commercially available from sources including BioCBD+, Active CBD oil, RSHO™ (Medical Marijuana, Inc., Poway, Calif.), and Ethos Innovates™ (One LED Corp, Bainbridge Island, Wash.). Commercially available THC cannabis extracts include Zoots™ (Natural Extractions, LLC, University Place, Wash.); Dixie Elixirs, Marijuana Drops (Marijuana Market), and Ethos Innovates.

In particular embodiments, primary cannabinoids can be purchased as non-decarboxylated cannabis extracts (containing THCA and/or CBDA instead of THC and/or CBD), and can be decarboxylated during formulation. The relative cannabinoid content of a cannabis strain is typically preserved during extraction (e.g. CO₂ or BHO extraction). Extracts sourced from a single strain can be useful for mimicking the entourage effects of a particular strain by providing the strain's natural repertoire of primary and additional cannabinoids. Non-decarboxylated cannabis extracts are commonly available for a wide variety of cannabis strains, such as Sour Diesel, Super Lemon Haze, Pure Kush, Charlotte's Web™, and Durban Poison.

In particular embodiments, cannabinoids that are provided in non-decarboxylated cannabis extracts are decarboxylated prior to formulating compositions for oral delivery. Decarboxylation of cannabinoids in a cannabis extract can be performed by heating the cannabis extract in a boiling water bath for 90 minutes. In particular embodiments, decarboxylation of the cannabinoids is performed prior to mixing with entourage-restoring molecules, because certain entourage-restoring molecules can be destroyed by heat.

Entourage-restoring molecules. In particular embodiments, entourage-restoring molecules can be obtained from commercially available sources. Many terpenes and flavonoids, such as linalool, β-myrcene, α-pinene, β-pinene, caryophyllene, quercetin, and apigenin are commercially available from various sources. Examples of companies that provide food grade terpenes and flavonoids include Sigma Aldrich, True Terpenes, and NHR Organic Oils. Examples of companies that provide aroma and flavor conferring volatiles include Sigma Aldrich, Eastman Chemical Company, Foodchem International Corporation, and Aurochemicals.

In particular embodiments, the entourage-restoring molecules are synthetically produced.

In particular embodiments, the cannabinoids can be synthetically produced. Examples of techniques for synthetic production of cannabinoids can be found in US2016/0355853; JP2016/509842; Petrzilka et al., Hely Chim Acta. 1967. 50(2):719-723; Kobayashi et al., Org Lett. 2006. 8(13):2699-2702; and Mechoulam & Gaoni, J Am Chem Soc. 1965. 87(14):3273-3275.

In particular embodiments, terpenes can be synthetically produced. Examples of techniques for synthetic production of terpenes can be found in US2004/0161819; and W02006134523. In particular embodiments, organisms can be genetically altered to overexpress particular terpenes and the terpenes can be isolated from the organism. Examples of techniques for obtaining terpenes from a genetically modified organism can be found in W020061111924 and US2010/0297722.

In particular embodiments, the flavonoids can be synthetically produced. Exemplary techniques to synthesize flavonoids can be found in Mamoalosi & Van Heerden, Molecules. 2013. 18: 4739-4765 and Wagner & Farkas, The Flavonoids. Chapter: Synthesis of Flavonoids. 1975. 127-213. Springer.

In particular embodiments, the entourage-restoring molecules are derived from vegetable matter. Vegetable matter is matter produced by a plant and includes any whole plant or plant part (e.g., bark, wood, leaves, stems, roots, flowers, fruits, seeds, or parts thereof) and/or exudates or extracts thereof. In particular embodiments, the compositions can include botanical products. Botanical products can include plant materials, algae, macroscopic fungi, and/or combinations thereof. In particular embodiments, the compositions include a mixture of various types of vegetable matter.

In particular embodiments, the entourage-restoring molecules can be prepared by pulverization, decoction, expression, and extraction of a starting plant product. The term “extract” can include all of the many types of preparations containing some or all of the active ingredients found in the relevant plants. Extracts may be produced by cold extraction techniques using a variety of different extraction solvents including water, fatty solvents (such as olive oil), and alcoholic solvents (e.g. 70% ethanol). Cold extraction techniques are typically applied to softer parts of the plant such as leaves and flowers, or in cases wherein the desired components of the plant are heat-labile (e.g., terpene) or have a low boiling point (e.g., volatiles). Alternatively, the aforementioned solvents may be used to produce extracts of the desired plants by a hot extraction technique, wherein said solvents are heated to a high temperature, the precise value of said temperature being dependent on the properties of the chosen solvent, and maintained at that temperature throughout the extraction process. Hot extraction techniques are more commonly applied to the harder, tougher parts of the plant, such as bark, woody branches and larger roots. In some cases, sequential extractions can be performed in more than one solvent, and at different temperatures. The plant extract may be used in a concentrated form. Alternatively, the extract may be diluted as appropriate to its intended use.

Additional procedures for producing plant extracts (including hot extraction, cold extraction and other techniques) are described in publications including “Medicinal plants: a field guide to the medicinal plants of the Land of Israel (in Hebrew), author: N. Krispil, Har Gilo, Israel, 1986” and “Making plant medicine, author: R. Cech, pub. by Horizon Herbs, 2000”.

In particular embodiments, the additional cannabinoids (non-THC, non-CBD cannabinoids), can be provided in a THC- and/or CBD-containing cannabis extract. Cannabis extracts (e.g. CO2 or BHO extracts) can be rich in cannabinoids and preserve the cannabinoid content of the cannabis strain used for extraction. Cannabinoid-rich cannabis extracts are commercially available from a variety of sources.

In particular embodiments, terpenes, flavonoids, and/or aroma and flavor conferring volatiles can be extracted from plants. Exemplary techniques for extracting terpenes from plants can be found in Breitmaier, Terpenes: Flavors, Fragrance, Pharmaca, Pheromones. Ch. 10. 2006. John Wley & Sons, WO2013174854 and CN101439074. An exemplary technique for obtaining a flavonoid-rich plant extract can be found in Victorio et al., Ecl. Quinn. 2009. 34(1):29-24. Techniques for extracting aroma and flavor conferring volatile compounds include cold-pressing and ethanol extraction.

In particular embodiments, the entourage-restoring molecules are obtained from extracts of plants other than cannabis. In particular embodiments, entourage-restoring molecules are obtained from any plant that produces the desired molecule. For example, vitexin is a cannabis-derived flavonoid that is also found in the plants hawthorn and passionflower and therefore vitexin can be obtained from hawthorn or passionflower extract.

Restoring Entourage Effects by Combining Cannabis-Derived Molecules. As indicated, in particular embodiments compositions with restored entourage effects are created by combining one or more primary cannabinoids (such as THC and/or CBD) with one or more entourage-restoring molecules.

In particular embodiments, the relative amount of each cannabis-derived molecule in the composition can be chosen to mimic the entourage effects of a particular cannabis strain. Cannabis strains can be tested to quantify primary cannabinoids and entourage molecules of a strain. The quantities of various cannabis-derived molecules present in a cannabis sample can be determined by analytical laboratory techniques, such as mass spectrometry, gas chromatography, or high performance liquid chromatography. Chemical profiling of cannabis strains is routinely performed by commercial testing laboratories, such as Steep Hill Labs, Inc., The Werc Shop, SC Labs and Analytical 360. Examples of terpenes quantified by commercial cannabis profiling labs include limonene, β-myrcene, caryophyllene, α-pinene, β-pinene, bisabolol, humulene, linalool, and terpinolene. Cannabinoids analyzed by commercial cannabis testing laboratories include THC, CBD, CBV, THCA, THCV, CBN, CBDA, CBL and CBG.

In particular embodiments, the primary cannabinoids and the entourage molecules are combined at a ratio that mimics their ratio in a particular cannabis strain, as measured through analytical testing. For example, compositions can be created to mimic the entourage effects of the strain Sour Diesel, for which a cannabinoid and terpene analysis is publicly availably (as a Strain Fingerprint®, Steep Hill Labs, Inc., Oakland Calif.). Sour Diesel can contain an average of 20% THC, 0.2% CBD, 0.5% CBG, 0.3% CBL, 0.3% β-myrcene, 0.3% limonene, and 0.25% caryophyllene. Therefore, a composition with restored Sour Diesel entourage effects can be created by combining 100 mg THC, 1 mg CBD, 2.5 mg CBG, 1.5 mg CBL, 1.5 mg β-myrcene, 1.5 mg limonene, and 1.25 mg caryophyllene, which mimics the strain's relative concentration of each of these components, providing a restored entourage effect compared to an otherwise equivalent composition lacking one or more of the entourage-restoring molecules. Other cannabis strains with publicly available cannabinoid and terpene analyses include Super Lemon Haze, Agent Orange, Berry While, Blue Dream, Cherry Pie, Durban Poison, Grape Ape, and Purple Kush.

In particular embodiments, the total concentration of primary cannabinoid(s) (e.g., THC and/or CBD) in the composition can be at 1 ug/ml or ug/mg, 10 ug/ml or ug/mg, 50 ug/ml or ug/mg, 100 ug/ml or ug/mg, 200 ug/ml or ug/mg, 300 ug/ml or ug/mg, 400 ug/ml or ug/mg, 500 ug/ml or ug/mg, 600 ug/ml or ug/mg, 700 ug/ml or ug/mg, 800 ug/ml or ug/mg, 900 ug/ml or ug/mg, or 950 mg/ml or mg/mg.

In particular embodiments, the ratio of the primary cannabinoids to each entourage-restoring molecule in the composition can be 1000:1, 500:1, 200:1, 100:1, 50:1, 20:0, 10:1, 1:1, 0.2:1, or 0.1:1. For example, a formulation with 5 mg CBD and 5 mg THC (10 mg totally primary cannabinoids) with 1 mg β-myrcene would have a 10:1 primary cannabinoid: β-myrcene ratio. In particular embodiments, the ratio of the primary cannabinoids to any entourage-restoring molecule can be chosen based on their ratio in any cannabis strain.

Formulating Compositions with a Carrier to Provide Fast-Acting Delivery. Particular embodiments include entourage effect-restored compositions prepared as fast-acting oral formulations. Exemplary oral formulations include capsules, coated tablets, edibles, elixirs, emulsions, gels, gelcaps, granules, gums, juices, liquids, oils, pastes, pellets, pills, powders, rapidly- dissolving tablets, sachets, semi-solids, sprays, solutions, suspensions, syrups, tablets, tinctures, etc.

Liquid preparations for oral administration may take the form of, for example, tinctures, solutions, syrups or suspensions, or they may be presented as a dry product for reconstitution with water or other suitable vehicles before use.

Exemplary formulation methods. Suspension formulation. In particular embodiments a composition with restored entourage effects and one or more N-acylated fatty amino acids are combined in water, an aqueous/organic solvent mixture or an organic solvent mixture. The resulting blend can be stirred to effect suspension.

Solution formulation. In particular embodiments a composition with restored entourage effects and one or more N-acylated fatty amino acids are combined in an aqueous/organic solvent mixture. The resulting blend is stirred vigorously for an hour. If solution is incomplete, a surfactant can be added and stirring can be continued to prepare the final formulation.

Gelcap formulation. In particular embodiments a suspension formulation or solution formulation can be filled into a gelcap to contain up to 1 g of a composition. The gelcap can be treated with an enteric coating or used without a coating.

Tablet/capsule formulation. The solution formulation and/or the suspension formulation can be dried by evaporation, lyophilization, or spray drying. The resultant dry product can be combined with tableting excipients and compressed into tablets or caplets to contain up to 1 g of the composition. Alternatively, the dry product can be filled into capsules.

In particular embodiments, the fast-acting oral formulations include tinctures. Tinctures are extracts or drugs dissolved in a solution of alcohol or alcohol and water. In particular embodiments, tinctures can be made by mixing a carrier and a composition with restored entourage effects with 20%-99% aqueous ethanol or 100% ethanol.

In particular embodiments the fast-acting oral formulations include edibles. Edibles refer to any product that can be consumed as a food or a drink. In some cases, edibles can be made by infusion of formulations into foodstuff. Examples of edible foods appropriate for use include candy, a candy bar, bread, a brownie, cake, cheese, chocolate, cocoa, a cookie, gummy candy, a lollipop, a mint, a pastry, peanut butter, popcorn, a protein bar, rice cakes, yogurt, etc. While technically not edible, gums can also be used. Examples of edible drinks include alcohol, beer, juice, flavored milk, flavored water, liquor, milk, punch, a shake, soda, tea, and water. In particular embodiments, edibles are made by combining the formulations with ingredients used to make an edible.

In particular embodiments, the primary cannabinoid(s), entourage-restoring molecule(s), and carrier(s) can be added separately to the edibles. In particular embodiments, a butter or oil can be heated (simmered for 3-4 hours) with a cannabis extract or components of a cannabis plant (e.g. flowers, stems, and/or leaves) to decarboxylate cannabinoids, and the cannabinoid-infused oil or fat can be used as an ingredient in the edible. Examples of food-grade fats and oils include butters and plant-based oils, such as coconut oil, grape seed oil, olive oil, palm oil, papaya seed oil, peanut oil, sesame oil, sprouted wheat oil, wheat germ oil, or any combination thereof. In particular embodiments, heat-sensitive components of the compositions (e.g. terpenes) can be added after decarboxylation or after cooking (e.g. by infusion).

Particular embodiments include swallowable formulations. Swallowable formulations are those that do not readily dissolve when placed in the mouth and may be swallowed whole without chewing or discomfort. U.S. Pat. Nos. 5,215,754 and 4,374,082 describe methods for preparing swallowable formulations. In particular embodiments, swallowable formulations may have a shape containing no sharp edges and a smooth, uniform and substantially bubble free outer coating.

To prepare swallowable formulations, each of the ingredients may be combined in intimate admixture with a suitable carrier according to conventional compounding techniques. In particular embodiments of the swallowable formulations, the surface of the compositions may be coated with a polymeric film. Such a film coating has several beneficial effects. First, it reduces the adhesion of the compositions to the inner surface of the mouth, thereby increasing the subject's ability to swallow the compositions. Second, the film may aid in masking the unpleasant taste of certain ingredients. Third, the film coating may protect the compositions from atmospheric degradation. Polymeric films that may be used in preparing the swallowable formulations include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In particular embodiments, the oral formulations may include chewable formulations. Chewable formulations are those that have a palatable taste and mouthfeel, are relatively soft and quickly break into smaller pieces and begin to dissolve after chewing such that they are swallowed substantially as a solution.

U.S. Pat. No. 6,495,177 describes methods to prepare chewable formulations with improved mouthfeel. U.S. Pat. No. 5,965,162, describes kits and methods for preparing comestible units which disintegrate quickly in the mouth, especially when chewed.

In order to create chewable formulations, certain ingredients should be included to achieve the attributes just described. For example, chewable formulations should include ingredients that create pleasant flavor and mouthfeel and promote relative softness and dissolvability in the mouth. The following discussion describes ingredients that may help to achieve these characteristics.

Sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Fondant or gums such as gelatin, agar, arabic gum, guar gum, and carrageenan may be added to improve the chewiness of the formulations. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of chewable formulations. By “enhance the texture” it is meant that the alkyl polysiloxane improves one or more of the stiffness, the brittleness, and the chewiness of the chewable formulation, relative to the same preparation lacking the alkyl polysiloxane. By “enhance the mouthfeel” it is meant that the alkyl polysiloxane reduces the gritty texture of the chewable formulation once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the back bone. Depending upon their grade, they can further include silica gel. Alkyl polysiloxanes are generally viscous oils. Exemplary alkyl polysiloxanes that can be used in swallowable, chewable or dissolvable formulations include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than 90.5% by weight (CH₃)₃—Si{OSi(CH₃)₂}CH₃ in admixture with 4.0% to 7.0% by weight SiO₂.

To prevent the stickiness that can appear in some chewable formulations and to facilitate conversion of the active ingredients to emulsion or suspension upon taking, the formulations may further include emulsifiers such as glycerin fatty acid ester, sorbitan monostearate, sucrose fatty acid ester, lecithin and mixtures thereof. In particular embodiments, one or more of such emulsifiers may be present in an amount of 0.01% to 5.0%, by weight of the administered formulations. If the level of emulsifier is lower or higher, in particular embodiments, an emulsification cannot be realized, or wax value will rise.

In particular embodiments, the oral formulations include one or more carriers (as previously described) and one or more excipients. For clarity, carriers contribute to providing an administration benefit. Excipients can, but need not, contribute to an administration benefit.

Excipients are commercially available from companies such as Aldrich Chemical Co., FMC Corp, Bayer, BASF, Alexi Fres, Wtco, Mallinckrodt, Rhodia, ISP, and others.

Exemplary excipient classes include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants may be included in the formulations to impart color to the formulation. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents can enhance the granulation of formulations. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycine.

Disintegrants also may be included in the formulations in order to facilitate dissolution. Disentegrants, including permeabilizing and wicking agents, are capable of drawing water or saliva up into the oral formulations which promotes dissolution from the inside as well as the outside of the oral formulations. Such disintegrants, permeabilizing and/or wicking agents that may be used include starches, such as corn starch, potato starch, pre-gelatinized and modified starches thereof, cellulosic agents, such as Ac-di-sol, montmorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, β-cyclodextrins, polymers, such as carbopol, and cellulosic agents, such as hydroxymethylcellulose, hydroxypropylcellulose and hydroxyopropylmethylcellulose. Dissolution of the oral formulations may be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Exemplary emulsifiers include acacia and lecithin.

Flavorants are natural or artificial compounds used to impart a pleasant flavor and often odor to oral formulations. Exemplary flavorants include, natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that may be used include natural berry extracts and natural mixed berry flavor, as well as citric and malic acid.

Glidants improve the flow of powder blends during manufacturing and minimize formulation weight variation. Exemplary glidants include silicon dioxide, colloidal or fumed silica, magnesium stearate, calcium stearate, stearic acid, cornstarch, and talc.

Lubricants are substances used in formulations that reduce friction during formulation compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

Exemplary preservatives include methyl p-hydroxybenzoates, propyl p-hydroxybenzoates, and sorbic acid.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

In addition to those described above, any appropriate fillers and excipients may be utilized in preparing the swallowable, chewable and/or dissolvable formulations or any other oral formulation described herein so long as they are consistent with the described objectives.

Additional information can be found in WADE & WALLER, HANDBOOK OF PHARMACEUTICAL EXCIPIENTS (2nd ed. 1994) and Remington's Pharmaceutical Sciences, 18th Ed. Mack Printing Company, 1990.

In particular embodiments, the composition with restored entourage effects can be preset in the formulation at a concentration of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; or at least 95% w/v or w/w of oral formulation.

In particular embodiments, 10 g of the composition may be used in 150 ml of water. This may give an effective composition concentration of between 1 and 99% (w/w), between 2 and 80% (w/w), and between 5 and 50% (w/w) in the formulation.

Formulations can be prepared to meet sterility, pyrogenicity, general safety, and purity standards as required by U.S. F.D.A. and/or other relevant foreign regulatory agencies.

Oral formulations can be individually wrapped or packaged as multiple units in one or more packages, cans, vials, blister packs, or bottles of any size. Doses are sized to provide therapeutically effective amounts.

Particular embodiments utilize one or more plant-derived molecules (e.g., a cannabinoid) with low solubility, or very low solubility. In particular embodiments, low solubility can refer to less than 0.2 mg/ml solubility in water or an aqueous solution, or less than 0.1 mg/ml solubility in water or an aqueous solution. Particular embodiments utilize plant-derived molecules that are essentially water insoluble. In particular embodiments, solubility in water is defined as low to insoluble by the United States pharmacopeia (USP 32) according to the amount of water necessary for the dissolution of one part of solute: Low solubility: 100 to 1000 parts of water necessary for dissolution of one part of solute; very low solubility: 1000 to 10,000 parts of water necessary; essentially water insoluble more than 10,000 parts of water necessary. At a basic pH, however, SNAC and other modified amino acids and FA-aas described herein are water soluble. Thus, the administration benefits, as described herein could not be reasonably predicted and are unexpected.

Methods to Provide Physiological Effects by Delivering Fast-Acting Formulations with Restored Entourage Effects. Formulations disclosed herein can be used to treat subjects (humans, veterinary animals (dogs, cats, reptiles, birds, etc.), livestock (horses, cattle, goats, pigs, chickens, etc.), and research animals (monkeys, rats, mice, fish, etc.)). Treating subjects includes providing effective amounts. Effective amounts include prophylactic treatments, therapeutic treatments, and/or effective amounts.

An “effective amount” is the amount of a formulation necessary to result in a desired physiological change in a subject. Effective amounts are often administered for recreational or research purposes. Research effective amounts disclosed herein can reduce pain perception in an animal model (neuropathic pain, acute pain, visceral pain), stimulate appetite in an animal model, reduce seizures (e.g., epileptic seizures) in an animal model, reverse bone loss in an animal model, relieve migraine (vasoconstrict cranial blood vessels) in an animal model, treat addiction in an animal model, reduce anxiety in an animal model, and/or reduce symptoms of asthma in an animal model. Recreational effective amounts can be used to elicit a desired physiological change that is not intended to provide medicinal or nutritional value.

A “prophylactic treatment” includes a treatment administered to a subject who does not display signs or symptoms of a disease or nutritional deficiency, or displays only early signs or symptoms of a disease or nutritional deficiency, such that treatment is administered for the purpose of diminishing, preventing, or decreasing the risk of developing the disease or nutritional deficiency further. Thus, a prophylactic treatment functions as a preventative treatment against the development of diseases or nutritional deficiencies.

As one example of a prophylactic treatment, a formulation disclosed herein can be administered to a subject who is at risk of developing a migraine headache. An effective prophylactic treatment of a migraine headache occurs when the number of migraines per month experienced by a subject is reduced by at least 10% or in particular embodiments, by 25%.

As another example of a prophylactic treatment, a formulation disclosed herein can be administered to a subject who is at risk of having an epileptic seizure. An effective prophylactic treatment of epileptic seizures occurs when the number of seizures per month is reduced by at least 10% or in particular embodiments, by 25%.

As another example of a prophylactic treatment, a formulation disclosed herein can be administered to a subject who is at risk of suffering from neuropathic pain. An effective prophylactic treatment of neuropathic pain occurs when the occurrence of the neuropathic pain is reduced by at least 10%, or in particular embodiments, by 25% as measured by a standard subjective or objective pain assessment.

As another example of a prophylactic treatment, a formulation disclosed herein can be administered to a subject who is at risk of developing breakthrough pain. An effective prophylactic treatment of breakthrough pain occurs when the occurrence of breakthrough pain is reduced by 10%, and in particular embodiments, by 25% by a standard subjective or objective pain assessment.

As another example of a prophylactic treatment, a formulation disclosed herein can be administered to a subject who is at risk of developing chemotherapy induced nausea and vomiting (CINV). An effective prophylactic treatment of CINV occurs when CINV is reduced by 10%, and in particular embodiments, by 25% measured by a standard subjective or objective CINV assessment.

As an example of a prophylactic treatment of a nutritional deficiency, a formulation disclosed herein can be administered to a subject who is at risk of developing rickets from insufficient vitamin C, anemia from insufficient dietary iron, and/or bone loss from insufficient calcium. An effective prophylactic treatment of these conditions occurs when the conditions are avoided or delayed due to nutritional supplementation with an oral formulation disclosed herein.

A “therapeutic treatment” includes a treatment administered to a subject who has a disease or nutritional deficiency and is administered to the subject for the purpose of curing or reducing the severity of the disease or nutritional deficiency.

As one example of a therapeutic treatment, a formulation disclosed herein can be administered to a subject who has a migraine headache. An effective therapeutic treatment of the migraine headache occurs when the severity of the headache is reduced or relieved completely and/or the headache resolves more quickly measured by a standard subjective or objective headache assessment.

Another example of a therapeutic treatment includes administration of a formulation disclosed herein to a subject experiencing CINV. A therapeutic treatment of CINV occurs when the vomiting is reduced or ceases (or ceases more quickly) and the nausea is relieved measured by a standard subjective or objective CINV assessment.

Another example of a therapeutic treatment, includes administration of a formulation disclosed to a subject who has osteoporosis. An effective therapeutic treatment of osteoporosis occurs when bone density has increased by 10% and in particular embodiments, by 25%.

Another example of a therapeutic treatment includes administration of a formulation disclosed herein to a subject who has anxiety. An effective therapeutic treatment of anxiety occurs when the severity of the anxiety is reduced or relieved completely and/or more quickly measured by a standard subjective or objective anxiety assessment.

Another example of a therapeutic treatment includes administration of a formulation disclosed herein to a subject who has multiple sclerosis. An effective therapeutic treatment of multiple sclerosis occurs when the score in a standard walk test improves by 10% and in particular embodiments, by 25%.

As one example of a therapeutic treatment of a nutritional deficiency, a formulation disclosed herein can be administered to a subject who has rickets from insufficient vitamin C, anemia from insufficient dietary iron, and/or bone loss from insufficient calcium. An effective therapeutic treatment of these conditions occurs when the conditions are reduced or resolved due to nutritional supplementation with a formulation disclosed herein.

Therapeutic treatments can be distinguished from effective amounts based on the presence or absence of a research component to the administration. As will be understood by one of ordinary skill in the art, however, in human clinical trials effective amounts, prophylactic treatments and therapeutic treatments can overlap.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by the subject, a physician, veterinarian, or researcher taking into account parameters such as physical, physiological and psychological factors including target, body weight, condition, previous or concurrent therapeutic interventions, and/or idiopathy of the subject.

Useful doses can range from 0.1 to 5 μg/kg or from 0.5 to 1 pg /kg. In other non-limiting examples, a dose can include 1 pg /kg, 5 pg /kg, 10 pg /kg, 15 pg /kg, 20 pg /kg, 25 pg /kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In other non-limiting examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg or more.

In particular embodiments, useful doses include weight of an active ingredient (e.g., a primary cannabinoid or an entourage-restoring molecule) per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, or more of an active ingredient per body weight of a subject.

In particular embodiments, useful doses include weight of carrier (e.g., SNAC) per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or more of carrier per body weight of a subject.

In particular embodiments, total dose volume can range from 0.25 mL to 30 mL or from 0.5 mL to 20 mL. In particular embodiments, a total dose volume can include 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, or more.

Dose concentration can be expressed as weight of an active ingredient per dose volume (e.g., mg active pharmaceutical ingredient (API)/mL). In particular embodiments, dose concentration can range from 1 mg/mL to 100 mg/mL or from 5 mg/mL to 50 mg/mL. In particular embodiments, a dose concentration can include 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL,17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more.

Dose concentration can be expressed as weight of carrier (e.g., SNAC) per dose volume (e.g., mg SNAC/ mL). In particular embodiments, dose concentration can range from 1 mg/mL to 500 mg/mL or from 50 mg/mL to 300 mg/mL. In particular embodiments, a dose concentration can include 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, or more.

In particular embodiments, the ratio of carrier to active ingredient (w/w) can range from 1:1 to 100:1 or from 1:1 to 20:1. In particular embodiments, the ratio can include 1:1, 2:1, 3:1, 4:1, 5:1, 6:1, 7:1, 8:1, 9:1, 10:1, 11:1,12:1,13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, or more. In particular embodiments, the ratio can be 10:1.

In particular embodiments, the ratio of carrier to primary cannabinoid (w/w) can range from 1:1 to 100:1 or from 1:1 to 20:1. In particular embodiments, the ratio can include 1:1, 2:1, 3:1, 4:1, 5:1, 6:1, 7:1, 8:1, 9:1, 10:1, 11:1,12:1,13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, or more. In particular embodiments, the ratio can be 10:1. In particular embodiments, a ratio of a carrier to a primary cannabinoid (w/w) of between 1:1 and 100:1 may provide an administration benefit. In particular embodiments, a ratio of a carrier to a primary cannabinoid (w/w) of between 1:1 and 20:1 may provide an administration benefit.

In particular embodiments, the ratio of primary cannabinoid to entourage-restoring molecule can range from 1000:1 to 0.1:1. In particular embodiments, the ratio of primary cannabinoid to entourage-restoring molecule can be 1000:1, 500:1, 200:1, 100:1, 50:1, 20:0, 10:1, 1:1, 0.2:1, or 0.1:1.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

One or more formulations can be administered simultaneously or within a selected time window, such as within 10 minutes, 1 hour, 3 hour, 10 hour, 15 hour, 24 hour, or 48 hour time windows or when the complementary formulation is given within a clinically-relevant therapeutic window.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A fast-acting oral formulation including
   (i) one or more of THC, CBD, and/or analogs thereof,
   (ii) one or more entourage-restoring molecules and
   (iii) a carrier,
wherein the THC, CBD, and/or analogs thereof and the one or more entourage-restoring molecules are provided at ratios that mimic their natural ratios within a cannabis strain.
2. The fast-acting oral formulation of embodiment 1 including THC and CBD.
3. The fast-acting oral formulation of embodiment 2 wherein the ratio of THC:CBD can be 0.01- 100:1.
4. The fast-acting oral formulation of any of embodiments 1-3 wherein the entourage-restoring molecules are selected from one or more of additional cannabinoids, terpenes, flavonoids, and aroma and flavor conferring volatiles.
5. The fast-acting oral formulation of embodiment 4 wherein the additional cannabinoids are selected from one or more of Δ8-tetrahydrocannabinol (Δ8-THC), Δ11-tetrahydrocannabinol (Δ11-THC), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA).
6. The fast-acting oral formulation of any of embodiment 4 or 5 wherein the terpenes are selected from one or more of β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, sabinene and phellandrene.
7. The fast-acting oral formulation of any of embodiments 4-6 wherein the flavonoids are selected from one or more of cannaflavin A, cannaflavin B, cannaflavin C, vitexin, isovitexin, apigenin, kaempferol, quercetin, luteolin, cinnamaldehyde, and orientin.
8. The fast-acting oral formulation of any of embodiments 4-7 wherein the aroma and flavor conferring molecules are selected from one or more of 2-heptanone, methyl heptanoate, methyl salicylate, methyl anthranilate, and hexanal.
9. The fast-acting oral formulation of any of embodiments 1-8 wherein the one or more entourage-restoring molecules are each present in the formulation at a ratio of 0.1-100:1 entourage-restoring molecule: THC and/or CBD.
10. The fast-acting oral formulation of any of embodiments 1-9 wherein the carrier includes an N-acylated fatty amino acid or a salt thereof.
11. The fast-acting oral formulation of embodiment 10 wherein the N-acylated fatty amino acid includes one or more of Compounds I-XXXV (FIG. 2), or Compounds α-r (FIG. 3).
12. The fast-acting oral formulation of embodiment 10 wherein the N-acylated fatty amino acid is selected from monosodium-N-salicyloyl-8-aminocaprylate, disodium-N-salicyloyl-8-aminocaprylate, and N-(salicyloyl)-8- aminocaprylic acid.
13. The fast-acting formulation of embodiment 10 wherein the N-acylated fatty amino acid or a salt thereof includes

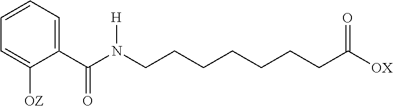

wherein X and Z are independently H, a monovalent cation, a divalent metal cation, or an organic cation.
14. The fast-acting formulation of embodiment 13, wherein X is H.
15. The fast-acting formulation of embodiment 13, wherein X is a monovalent cation such as sodium or potassium.
16. The fast-acting formulation of embodiment 13, wherein X is a metal cation such as calcium or magnesium.
17. The fast-acting formulation of embodiment 13, wherein X is an organic cation such as ammonium or tetramethylammonium.
18. The fast-acting formulation of any of embodiments 13-17, wherein Z is H.
19. The fast-acting formulation of any of embodiment 13-17, wherein Z is a monovalent cation such as sodium.
20. The fast-acting formulation of any of embodiment 13-17, wherein Z is a divalent cation such as calcium or magnesium.
21. The fast-acting formulation of embodiment 13, wherein X is H and Z is H.
22. The fast-acting formulation of embodiment 13, wherein X is H and Z is sodium.
23. The fast-acting formulation of embodiment 13, wherein X is sodium and Z is sodium.
24. The fast-acting formulation of any of embodiments 1-23 further including a surfactant, detergent, azone, pyrrolidone, glycol, or bile salt.
25. The fast-acting formulation of any of embodiments 1-24 wherein the composition with restored entourage effects includes one or more plant extracts.
26. The oral formulation of any of embodiments 1-25 wherein the oral formulation is swallowable or chewable.
27. The fast-acting oral formulation of any of embodiments 1-26 wherein the oral formulation is liquid or solid.
28. The fast-acting oral formulation of any of embodiments 1-27 wherein the oral formulation is a solution, suspension, gel, juice, oil, paste, emulsion, tincture or spray.
29. The fast-acting oral formulation of any of embodiments 1-28 wherein the oral formulation is a tablet, capsule, edible, pill, gelcap, granule, gum or sachet.
30. The fast-acting oral formulation of embodiment 1-29 wherein the formulation is flavored.
31. The fast-acting oral formulation of any of embodiments 1-30 including an effective amount of the formulation.
32. The fast-acting oral formulation of embodiment 31 wherein the effective amount is a therapeutic amount, a prophylactic amount, a research effective amount, or a recreationally effective amount.
33. The fast-acting oral formulation of embodiment 31 or 32 wherein the effective amount includes 0.1 mg-100 mg THC.
34. The fast-acting oral formulation of any of embodiments 30-33 wherein the effective amount includes 0.1 mg-100 mg CBD.
35. The fast-acting oral formulation of any of embodiments 10-34, wherein the N-acylated fatty amino acid is at a dose of 100-200 mg.
36. The fast-acting oral formulation of any of embodiments 10-35, wherein the N-acylated fatty amino acid or salt thereof is at a dose concentration of 100 mg/mL to 300 mg/mL.
37. The fast-acting oral formulation of any of embodiments 10-36, wherein the N-acylated fatty amino acid or salt thereof is at a dose concentration of 250 mg/mL.
38. The fast-acting oral formulation of any of embodiments 10-37, wherein the N-acylated fatty amino acid or salt thereof is at a dose of one to one hundred times the dose of the one or more cannabinoids.
39. A nutritional supplement including a formulation of any of embodiments 1-38 and i) a vitamin or a mineral, or ii) a vitamin and a mineral.
40. The nutritional supplement of embodiment 39 wherein the vitamin includes Vitamin A, Vitamin B1, Vitamin B6, Vitamin B12, Vitamin C, Vitamin D, Vitamin E, or Vitamin K.
41. The nutritional supplement of embodiment 39 or 40 wherein the mineral includes calcium, chromium, iodine, iron, magnesium, selenium or zinc.
42. A method of preparing a composition including (i) THC and/or CBD and/or an analog thereof and (ii) one or more entourage-restoring molecules, wherein the method includes adding an absorption enhancer to the composition and wherein the composition has a faster onset of action than an equivalent composition without an absorption enhancer.
43. The method of embodiment 42, wherein the absorption enhancer is an N-acylated fatty amino acid or a salt thereof.
44. The method of embodiment 43, wherein the N-acylated fatty amino acid includes one or more of Compounds I-XXXV (FIG. 2), or Compounds α-r (FIG. 3).
45. The method of embodiment 43, wherein the N-acylated fatty amino acid is selected from monosodium-N-salicyloyl-8-aminocaprylate, disodium-N-salicyloyl-8-aminocaprylate, and N- (salicyloyl)-8- aminocaprylic acid.
46. The method of embodiment 43, wherein the N-acylated fatty amino acid or a salt thereof includes

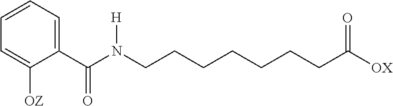

wherein X and Z are independently H, a monovalent cation, a divalent metal cation, or an organic cation.
47. The method of embodiment 46, wherein X is H.
48. The method of embodiment 46, wherein X is a monovalent cation such as sodium or potassium.
49. The method of embodiment 46, wherein X is a divalent metal cation such as calcium or magnesium.
50. The method of embodiment 46, wherein X is an organic cation such as ammonium or tetramethylammonium.
51. The method of any of embodiments 46-50, wherein Z is H.
52. The method of any of embodiments 46-50, wherein Z is a monovalent cation such as sodium or potassium.
53. The method of any of embodiments 46-50, wherein Z is a divalent cation such as calcium or magnesium.
54. The method of embodiment 46, wherein X is H and Z is H.
55. The method of embodiment 46, wherein X is H and Z is sodium.
56. The method of embodiment 46, wherein X is sodium and Z is sodium.
57. A method of treating a subject in need thereof including administering a therapeutically effective amount of a formulation of any of embodiments 1-38 to the subject thereby treating the subject in need thereof.
58. The method of embodiment 57 wherein the therapeutically effective amount provides an effective amount, a prophylactic treatment, and/or a therapeutic treatment.
59. The method of embodiment 57 or 58, wherein the N-acylated fatty amino acid provides an administration benefit.
60. The method of any of embodiments 59, wherein the administration benefit is a dose-dependent administration benefit.
61. The method of embodiment 60, wherein the dose-dependent administration benefit is at a dose of 100-200 mg.
62. The method of embodiment 60, wherein the dose-dependent administration benefit is at a dose concentration of 100 mg/mL to 300 mg/mL N-acylated fatty amino acid or salt thereof.
63. The method of embodiment 60, wherein the dose-dependent administration benefit is at a dose concentration of 1-500 mg/mL N-acylated fatty amino acid or salt thereof.
64. The method of embodiment 63, wherein the dose-dependent administration benefit is at a dose concentration of 250 mg/mL N-acylated fatty amino acid or salt thereof.
65. The method of any of embodiments 60-64, wherein the dose-dependent administration benefit of the N-acylated fatty amino acid or salt thereof is at a dose of one to one hundred times the dose of the one or more synthetic cannabinoids.
66. A method of reducing or eliminating one or more symptoms of a disease or disorder in a human subject,
   wherein said method includes delivering a therapeutically effective amount of a formulation of any of embodiments 1-38 to the subject, thereby reducing or eliminating one or more symptoms of the disease or disorder, and
   wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.

Example 1. Oral cannabinoid dosage form providing improved bioavailability and shortened time to onset of effect.

Considering the wealth of medical conditions potentially benefiting from cannabis therapy, a significant unmet need exists for a faster-acting product that provides improved bioavailability in an oral format. Current oral cannabis products include edibles and traditional pharmaceutical dosage forms that are challenged by low bioavailability, and prolonged time to onset of action. The present disclosure addresses the shortcomings of all of the currently available oral cannabis products to provide an improved time to onset of effect and improved bioavailability.

Onset and duration of action of orally administered cannabis/SNAC composition. This study was designed to assess the utility of SNAC in enabling a rapid-acting oral form of cannabis.

Selection of Participants. Six study participants were recruited to ingest cannabis compositions and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user- reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 200 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

TABLE 1
Scale Values for Reporting Euphoria and Dysphoria

| Scale Value | Description |
|---|---|
| 0 | No observed effect |
| 1-2 | Mild observed effect; possibly psychological |
| 3-4 | Definite but mild effect |
| 5-6 | Definite substantial effect |
| 7-8 | Strong effect |
| 9-10 | Intense effect |

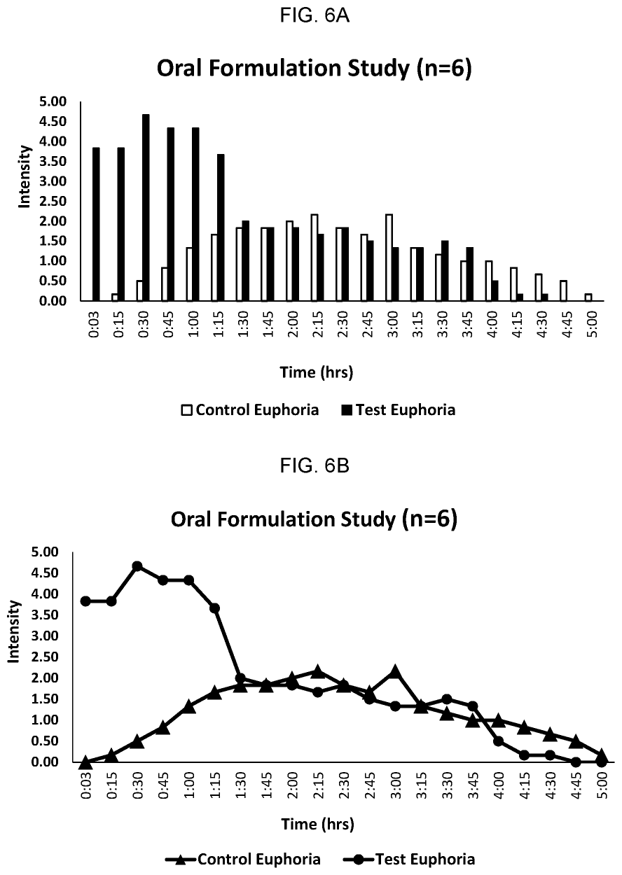

Results. The results shown below are the average scale values obtained for all six participants (also shown in FIGS. 6A and 6B).

TABLE 2
Control Experiment (n = 6)

| Actual | Time | Observed | Observed |
|---|---|---|---|
| Time | from Start | “Euphoria” | “Dysphoria” |
| 12:00 PM | 0:00 | (0-10) | (0-10) |
| 12:15 PM | 0:15 | 0.17 | 0.00 |
| 12:30 PM | 0:30 | 0.50 | 0.00 |
| 12:45 PM | 0:45 | 0.83 | 0.17 |
| 1:00 PM | 1:00 | 1.33 | 0.17 |
| 1:15 PM | 1:15 | 1.67 | 0.50 |
| 1:30 PM | 1:30 | 1.83 | 0.67 |
| 1:45 PM | 1:45 | 1.83 | 0.83 |
| 2:00 PM | 2:00 | 2.00 | 0.50 |
| 2:15 PM | 2:15 | 2.17 | 0.50 |
| 2:30 PM | 2:30 | 1.83 | 0.33 |
| 2:45 PM | 2:45 | 1.67 | 0.33 |
| 3:00 PM | 3:00 | 2.17 | 0.33 |
| 3:15 PM | 3:15 | 1.33 | 0.17 |
| 3:30 PM | 3:30 | 1.17 | 0.00 |
| 3:45 PM | 3:45 | 1.00 | 0.00 |
| 4:00 PM | 4:00 | 1.00 | 0.00 |
| 4:15 PM | 4:15 | 0.83 | 0.00 |
| 4:30 PM | 4:30 | 0.67 | 0.00 |
| 4:45 PM | 4:45 | 0.50 | 0.00 |
| 5:00 PM | 5:00 | 0.17 | 0.00 |

TABLE 3
Test Experiment (n = 6)

| | Time | | |
|---|---|---|---|
| Actual | from | Observed | Observed |
| Time | Start | “Euphoria” | “Dysphoria” |
| 12:00 PM | 0:00 | (0-10) | (0-10) |
| 12:03 PM | 0:03 | 3.83 | 0.67 |
| 12:15 PM | 0:15 | 3.83 | 0.67 |
| 12:30 PM | 0:30 | 4.67 | 0.83 |
| 12:45 PM | 0:45 | 4.33 | 0.50 |
| 1:00 PM | 1:00 | 4.33 | 0.50 |
| 1:15 PM | 1:15 | 3.67 | 0.67 |
| 1:30 PM | 1:30 | 2.00 | 0.17 |
| 1:45 PM | 1:45 | 1.83 | 0.17 |
| 2:00 PM | 2:00 | 1.83 | 0.00 |
| 2:15 PM | 2:15 | 1.67 | 0.00 |
| 2:30 PM | 2:30 | 1.83 | 0.00 |
| 2:45 PM | 2:45 | 1.50 | 0.00 |
| 3:00 PM | 3:00 | 1.33 | 0.17 |
| 3:15 PM | 3:15 | 1.33 | 0.17 |
| 3:30 PM | 3:30 | 1.50 | 1.00 |
| 3:45 PM | 3:45 | 1.33 | 0.00 |
| 4:00 PM | 4:00 | 0.50 | 0.00 |
| 4:15 PM | 4:15 | 0.17 | 0.00 |
| 4:30 PM | 4:30 | 0.17 | 0.00 |
| 4:45 PM | 4:45 | 0.00 | 0.00 |
| 5:00 PM | 5:00 | 0.00 | 0.00 |

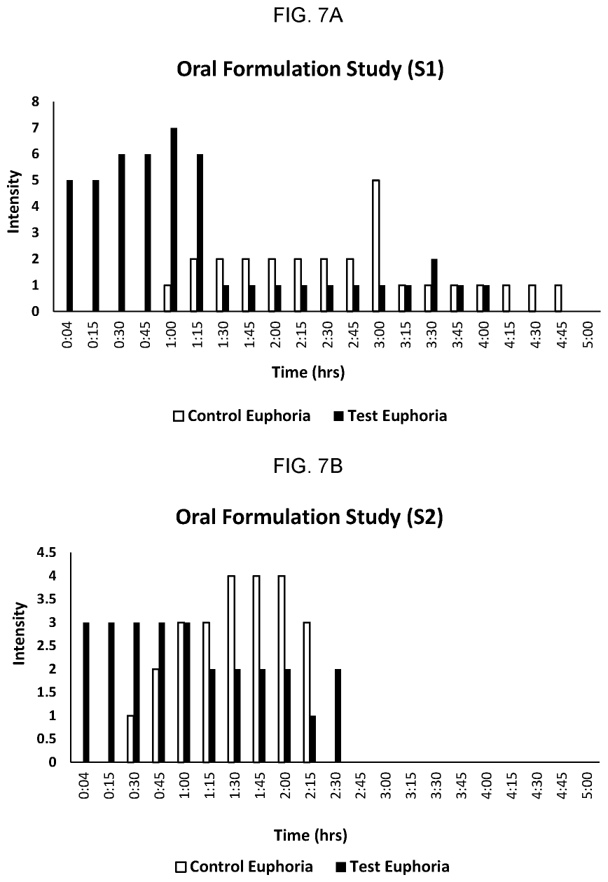

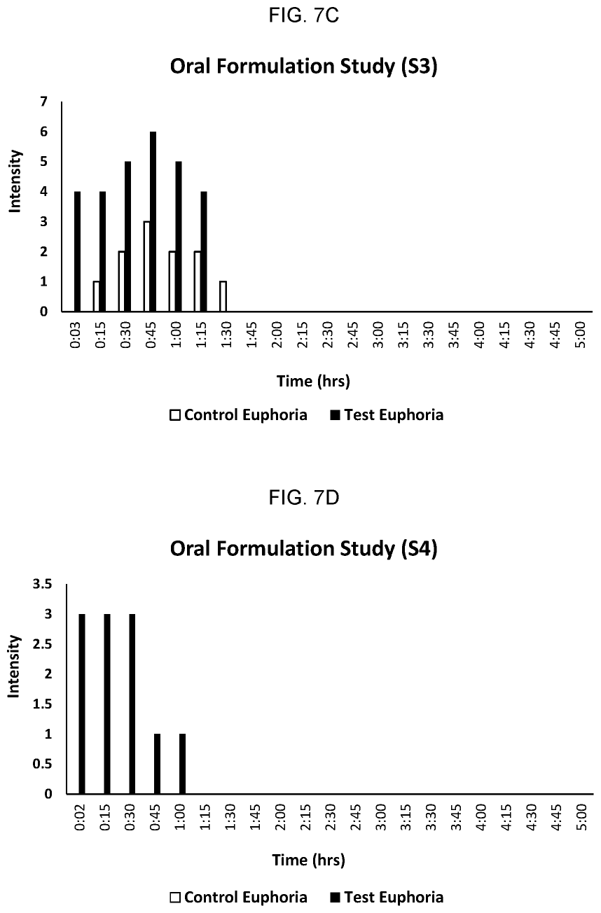

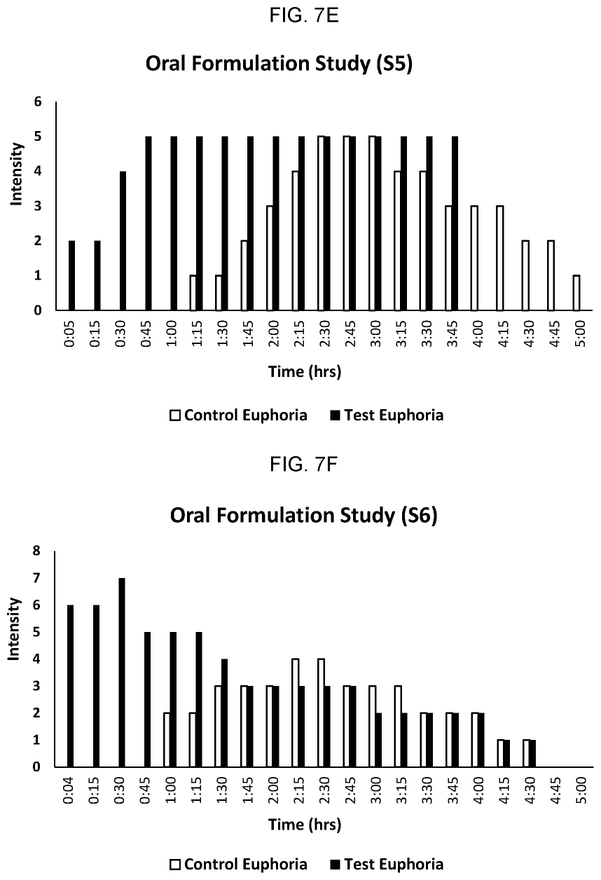

Onset: All six participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes (see FIGS. 7A-7F for individual participant results). By fifteen minutes post-ingestion, the average reported euphoria scale value was 3.8 for the cannabis/SNAC formulation (Test). In contrast, fifteen minutes after ingestion of the cannabis-only formulation (Control), the average reported euphoria scale value was 0.17 (see FIGS. 6A-6B for averages at each time-point).

Intensity: The average peak euphoria scale value after ingestion of the cannabis/SNAC formulation (Test) was 4.7, which occurred thirty minutes post-ingestion. In contrast, the highest average euphoria scale value after ingestion of the cannabis-only formulation (Control) was 2.2, which was at the two hour, fifteen minute time-point (see FIGS. 6A and 6B). Therefore, ingestion of the cannabis/SNAC formulation led to a higher peak intensity of euphoria, which occurred an average of one hour and forty-five minutes faster than when the cannabis-only formulation was ingested. The intensity of observed dysphoria was minimal for both the Test and Control, with a peak average scale value of 0.83 for both experiments.

Duration: The results indicate that the addition of an absorption enhancer does not shorten the duration of action of cannabis.

In summary, adding an absorption enhancer, such as SNAC, in an oral dosage formulation of cannabis provides faster onset of action and higher intensity of action at peak activity level of cannabis. Moreover, the absorption enhancer has no effect on the duration of action of cannabis,

Example 2. Onset and duration of action of orally administered cannabis/SNAC composition at a low SNAC dose. This study was designed to assess the utility of SNAC in enabling a rapid-acting oral form of cannabis at a low dose.

Selection of Participants. Three study participants were recruited to ingest cannabis compositions and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user- reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 100 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

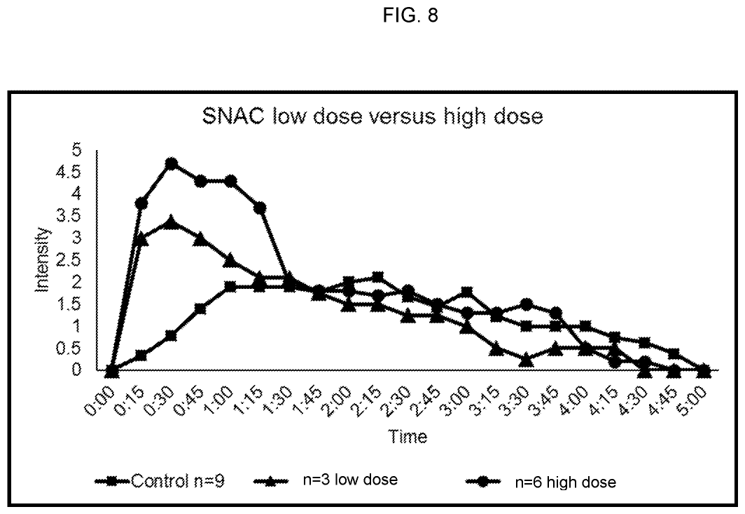

Results. The results are combined with the data from Example 1 and are reported for all participants in FIG. 8.

Onset: All three participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes. By fifteen minutes post-ingestion, the average reported euphoria scale value was 3.0 for the cannabis/SNAC formulation (Test). In contrast, fifteen minutes after ingestion of the cannabis-only formulation (Control), the average reported euphoria scale value was 0.25.

Intensity: The average peak euphoria scale value after ingestion of the cannabis/SNAC formulation (Test) was 3.4, which occurred thirty minutes post-ingestion. In contrast, the highest average euphoria scale value after ingestion of the cannabis-only formulation (Control) was 2.2, which was at the two hour, fifteen minute time-point. Compared to Example 1 where the SNAC dose was 200 mg, the participants in Example 2 ingested only 100 mg of SNAC combined with the same quantity of cannabis used in Example 1. This reduced quantity of SNAC resulted in a reduced cannabis effect demonstrating a clear dose-response relationship between observed cannabis effect (euphoria) and SNAC dose. Consistent with Example 1, ingestion of the cannabis/SNAC formulation led to a higher peak intensity of euphoria, which occurred an average of one hour and forty-five minutes faster than when the cannabis-only formulation was ingested.

Duration: The results indicate that the addition of an absorption enhancer does not shorten the duration of action of cannabis.

In summary, adding an absorption enhancer, such as SNAC, in an oral dosage formulation of cannabis provides faster onset of action and higher intensity of action at peak activity level of cannabis. Moreover, the absorption enhancer has no effect on the duration of action of cannabis. The varying quantity of SNAC produces a clear dose-response relationship between observed cannabis effect (euphoria) and SNAC dose.

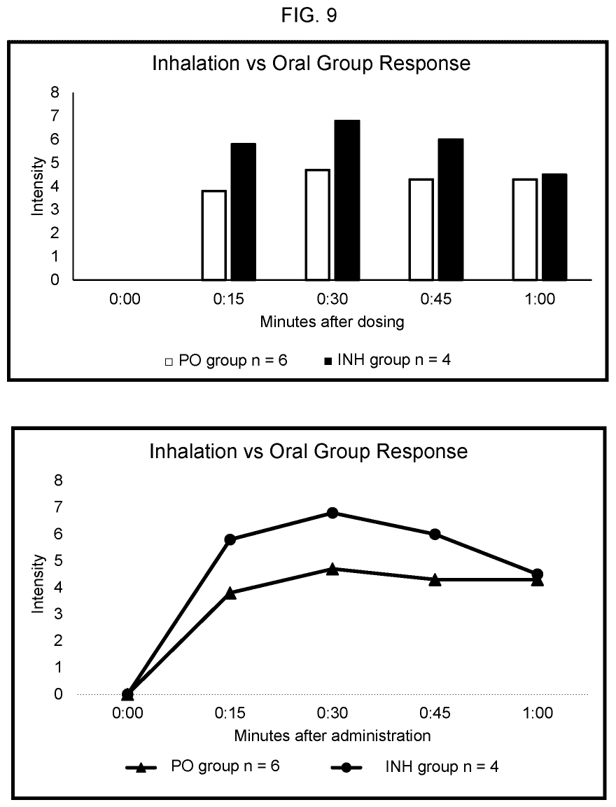

Example 3. Inhalation versus oral group response (FIG. 9). Comparison of the pharmacodynamic response to inhaled and oral cannabis measured as subject-reported euphoria. Both the oral and inhaled groups reported similar time to peak effect (15-30 minutes). This is very surprising because oral cannabis is traditionally characterized by a very slow time to peak effect (up to 4 hours).

Example 4. Summary of cannabis/SNAC oral rat pharmacokinetic (PK) study. The study was designed to characterize the pharmacokinetic profile of cannabis extract containing 56% THC/CBD in a 1:1 ratio (by weight) with and without the excipient, SNAC, following a single oral gavage administration to rats. In this study two doses of cannabis and SNAC and two ratios of cannabis to SNAC were tested. The experimental design is presented in Table 4 below.

TABLE 4
Experimental design.

| | | Extract¹ | SNAC | | | | |
|---|---|---|---|---|---|---|---|
| | | Dose | Dose | Dose | Dose | Dose | |
| Group | Group | Level | Level | Volume | Concentration² | Concentration³ | Number |
| Nos | Designation | (mg/kg) | (mg/kg) | (mL/kg) | (mg API/mL) | (mg SNAC/mL) | of Males |
| 1 | Excipient | 0 | 500 | 2 | 0 | 250 | 6 |
| | Control | | | | | | |
| 2 | Cannabis | 25 | 0 | 2 | 12.5 | 0 | 6 |
| | Control | | | | | | |
| 3 | Low Dose | 25 | 250 | 2 | 12.5 | 125 | 6 |
| 4 | Mid Dose | 25 | 500 | 2 | 12.5 | 250 | 6 |
| 5 | High Dose | 50 | 500 | 2 | 25 | 250 | 6 |

¹Extract contains 54% by weight (27% THC + 27% CBD) as the API (Active Pharmaceutical Ingredient)
²Dose of cannabis extract contains a mixture of THC:CBD in a ratio of 1:1 by weight
³SNAC dose is 10 times (THC + CBD) dose for groups 3 and 5 and 20 times for group 4.

Methods. Animals were dosed on Day 1 and a series of blood samples were collected over a period of 4 hours post dose for pharmacokinetic evaluation. Animals were euthanized following collection of their last blood sample.

Results. Following a single oral administration of cannabis extract containing THC/CBD in a 1:1 ratio combined with the absorption enhancing excipient (SNAC) at 25 mg extract/kg and 250 mg SNAC/kg (Group 3), 25 mg extract/kg and 500 mg SNAC/kg (Group 4), or 50 mg extract/kg and 500 mg SNAC/kg (Group 5), mean maximum concentration Cₘₐₓ ranged from 31.7 to 159.3 ng/mL for CBD and from 111.5 to 546.17 ng/mL for THC. The time to reach the mean maximum plasma concentration (Tₘₐₓ) ranged from 0.25 to 1 hour post dose for CBD and was reached at 1 hour post dose for the low and mid dose groups and at 2 hours post dose for the high dose group for THC. The AUC_{0-Tlast} ranged from 13.17 to 382.14 hr*ng/mL for CBD and from 170.64 to 1256.49 hr*ng/mL for THC.

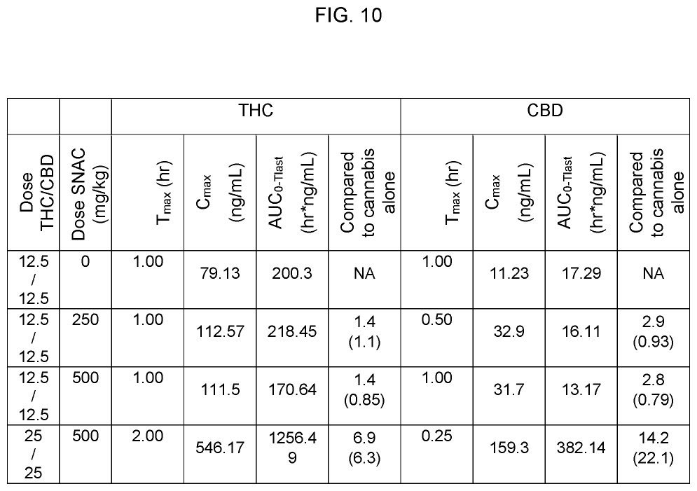

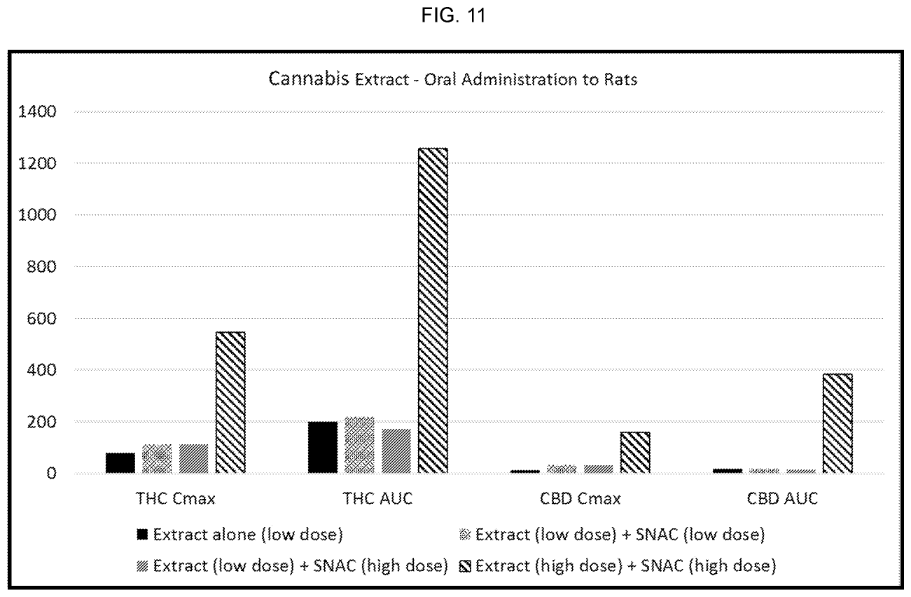

Over the dose range tested, Cₘₐₓ and AUC_{0-Tlast}, for THC was higher than for CBD. When administering the same cannabis extract (THC/CBD) dose (25 mg/kg total cannabinoid dose; 12.5 mg/kg THC/12.5 mg/kg CBD) with and without SNAC, for THC, a 1.4-fold Cₘₐₓ increase over cannabis alone was observed at SNAC doses of either 250 or 500 mg/kg. AUC was 1.1-fold greater in the 250 mg/kg SNAC group, but lower in the 500 mg/kg SNAC group, compared to the cannabis alone group. For CBD, 2.9-fold and 2.8-fold Cₘₐₓ increases over cannabis alone were observed at SNAC doses of either 250 or 500 mg/kg. AUC was lower in both groups, compared to the cannabis alone group. Increasing both the cannabis and SNAC doses 2-fold to 500 mg/kg SNAC and 50 mg/kg cannabis extract (25 mg/kg THC/25 mg/kg CBD), resulted in a 14.2-fold increase in the CBD Cₘₐₓ and a 6.9-fold increase in the THC Cₘₐₓ. AUC_{0-Tlast} for CBD and THC, were increased 22.1-fold and 6.3-fold, respectively (FIG. 10 and FIG. 11). Over the dose range tested, Cₘₐₓ and AUC_{0-Tlast}, for THC was higher than for CBD. When administering the same cannabis extract (THC/CBD) dose in the presence of SNAC (250 mg/kg or 500 mg/kg), both THC and CBD Cₘₐₓ were increased 1.4-fold and 2.8-fold, respectively, over the cannabis alone group. AUC_{0-Tlast} were comparable. This observation suggests that a cannabis to SNAC ratio of 10:1 facilitates an increase in Cₘₐₓ, but increasing the ratio to 20:1 provides no additional benefit. Increasing both the cannabis and SNAC doses by 2-fold resulted in THC and CBD Cₘₐₓ increases of 6.9-fold and 14.2-fold, respectively, over the cannabis alone group. AUC_{0-Tlast} for THC and CBD increased by 6.3-fold and 22.1-fold, respectively, over the cannabis alone group. This is a greater than expected increase based on the near linear dose response observed for oral cannabis (Information for Health Care Providers—Cannabis and the Cannabinoids; Health Canada February 2013). Overall, these data suggest that SNAC enhances cannabis absorption when administered to rats by oral gavage.

Example 5. Onset and duration of action of orally administered cannabis/NAC composition. This study was designed to assess the utility of the acid form of SNAC, N-[8-(2-hydroxybenzoyl) amino] caprylic acid (NAC), in enabling a rapid-acting oral form of cannabis.

Study Participant. One study participant was recruited to ingest cannabis compositions and record the onset, duration, and intensity of cannabis-induced euphoria and/or dysphoria. The study participant took part in two separate tests: 1) use of a control substance, which included cannabis concentrate oil in an herbal extract blend dissolved in aqueous ethanol, and 2) use of a test substance, which included the cannabis concentrate oil in an herbal extract blend dissolved in aqueous ethanol, as well as NAC.

Formulations. The selected cannabis concentrate oil is commercially available in a capsule and the contents of the capsule were provided to the participant in an ethanol solution. One capsule contains 9 mg CBD, 7.7 mg THC, herbal extract blend (Magnolia bark, Ashwagandha, Astragalus), and stearic acid (from vegetable oil), and the stated potency per capsule is: CBD 9.0 mg, THCA 0.0 mg and THC 7.6 mg. The formulation was selected because it provides a noticeable effect on user- reported “euphoria”, and the CBD content should ameliorate dysphoric effects if Test 2 delivers a very high dose of cannabinoids.

Methods. For the Control experiment, the participant mixed the cannabis concentrate with 15 ml (one tablespoon) of aqueous ethanol, and immediately swallowed the mixture.

For the Test experiment, the participant mixed the cannabis concentrate with 15 ml pre-mixed solution of aqueous ethanol and 100 mg NAC, and immediately swallowed the dissolved mixture.

For both the Control experiment and the Test experiment, the participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-5. Table 5 shows descriptions of euphoria and dysphoria levels for each scale value.

TABLE 5
Scale Values for Reporting Euphoria and Dysphoria

| Scale Value | Description |
|---|---|
| 0 | No observed effect |
| 1 | Mild observed effect; possibly psychological |
| 2 | Definite but mild effect |
| 3 | Definite substantial effect |
| 4 | Strong effect |
| 5 | Intense effect |

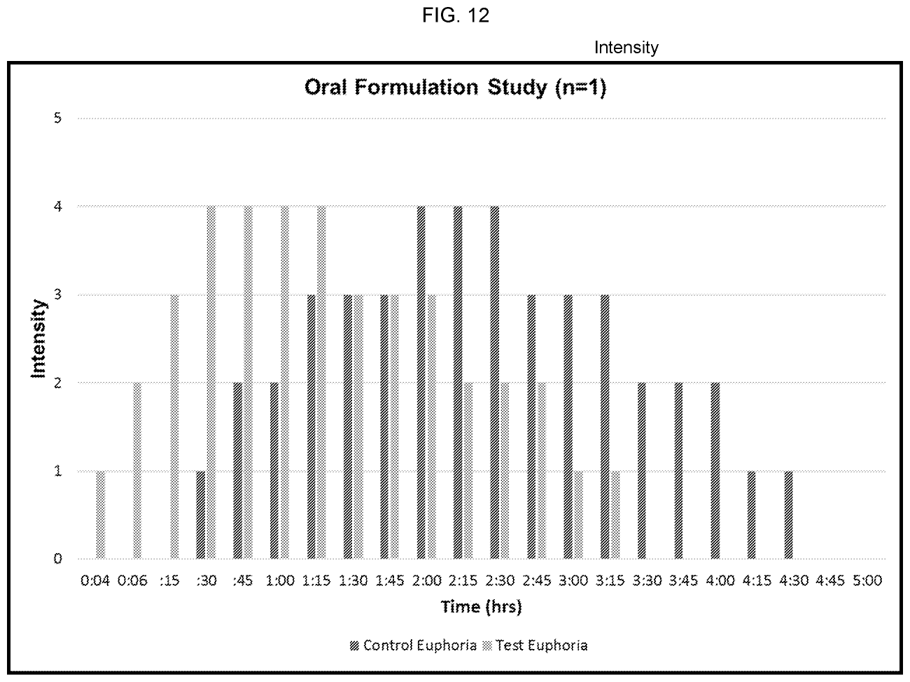

Results. The results shown below are scale values obtained for the participant in the control experiment (Table 6) and in the test experiment (Table 7). The values are plotted in FIG. 12.

TABLE 6
Control Experiment (n = 1)

| | Time from | Observed | Observed |
|---|---|---|---|
| Actual Time | Start | “Euphoria” | “Dysphoria” |
| 11:13 AM | 0:00 | (0-5) | (0-5) |
| 11:28 AM | :15 | 0 | 0 |
| 11:43 AM | :30 | 1 | 0 |
| 11:58 AM | :45 | 2 | 0 |
| 12:13 PM | 1:00 | 2 | 0 |
| 12:28 PM | 1:15 | 3 | 1 |
| 12:43 PM | 1:30 | 3 | 1 |
| 12:58 PM | 1:45 | 3 | 1 |
| 1:13 PM | 2:00 | 4 | 1 |
| 1:28 PM | 2:15 | 4 | 1 |
| 1:43 PM | 2:30 | 4 | 1 |
| 1:58 PM | 2:45 | 3 | 0 |
| 2:13 PM | 3:00 | 3 | 0 |
| 2:28 PM | 3:15 | 3 | 0 |
| 2:43 PM | 3:30 | 2 | 0 |
| 2:58 PM | 3:45 | 2 | 0 |
| 3:13 PM | 4:00 | 2 | 0 |
| 3:28 PM | 4:15 | 1 | 0 |
| 3:43 PM | 4:30 | 1 | 0 |
| 3:58 PM | 4:45 | 0 | 0 |
| 4:13 PM | 5:00 | 0 | 0 |

TABLE 7
Test Experiment (n = 1)

| | Time from | Observed | Observed |
|---|---|---|---|
| Actual Time | Start | “Euphoria” | “Dysphoria” |
| 11:20 AM | 0:00 | (0-5) | (0-5) |
| 11:24 AM | :04 | 1 | 0 |
| 11:26 AM | :06 | 2 | 0 |
| 11:35 AM | :15 | 3 | 1 |
| 11:50 AM | :30 | 4 | 0 |
| 12:05 PM | :45 | 4 | 0 |
| 12:20 PM | 1:00 | 4 | 0 |
| 12:35 PM | 1:15 | 4 | 0 |
| 12:50 PM | 1:30 | 3 | 0 |
| 1:05 PM | 1:45 | 3 | 0 |
| 1:20 PM | 2:00 | 3 | 0 |
| 1:35 PM | 2:15 | 2 | 0 |
| 1:50 PM | 2:30 | 2 | 0 |
| 2:05 PM | 2:45 | 2 | 0 |
| 2:20 PM | 3:00 | 1 | 0 |
| 2:35 PM | 3:15 | 1 | 0 |
| 2:50 PM | 3:30* | 0 | 0 |

*experiment ended

Onset: The participant reported euphoria within six minutes of ingesting the cannabis/NAC formulation (Test, Table 7 and FIG. 12). In contrast, the first time-point of euphoria reported by the participant after ingestion of the cannabis-only formulation (Control, Table 6 and FIG. 12) was forty-five minutes post-ingestion. By thirty minutes post-ingestion, the participant reported strong euphoria (scale value of 4) for the cannabis/NAC formulation. In contrast, thirty minutes after ingestion of the cannabis-only formulation, the participant only observed a mild effect that was possibly psychological (scale value of 1).

Intensity: The peak euphoria scale value after ingestion of both the cannabis-only (Control) and cannabis/NAC formulations (Test) was 4. However, peak intensity of euphoria was reached thirty minutes post-ingestion with the cannabis/NAC formulation (Test), whereas peak intensity of euphoria was reached two hours post-ingestion with the cannabis-only formulation (Control). Therefore, ingestion of the cannabis/NAC formulation led to a peak intensity of euphoria that occurred one hour and thirty minutes faster than when the cannabis-only formulation was ingested. The intensity of observed dysphoria was minimal for both the Test and Control, although the participant observed more mild dysphoria effects with the cannabis-only formulation (Control).

In summary, NAC, the acid form of SNAC, behaves similarly to SNAC when included in an oral dosage cannabinoid formulation, A cannabinoid/NAC formulation provides faster onset of action as compared to a cannabinoid-only formulation.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in an administration benefit when assessed in an animal model disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A fast-acting oral formulation comprising:
(i) a cannabinoid comprising THC and/or CBD,
(ii) an entourage-restoring molecule, and
(iii) N-[8-(2-hydroxybenzoyl) amino] caprylate (SNAC), wherein the cannabinoid and the entourage-restoring molecule are at a ratio of 1000:1, 500:1, 200:1, 100:1, 50:1, 20:0, 10:1, 1:1, 0.2:1, or 0.1:1, and
wherein the cannabinoid and the SNAC are at a ratio of between 1:1 and 100:1.

2. The fast-acting oral formulation of claim 1 comprising THC and CBD.

3. The fast-acting oral formulation of claim 2 wherein the ratio of THC:CBD is between 0.01:1 and 100:1.

4. The fast-acting oral formulation of claim 1 wherein the entourage-restoring molecule is selected from: an additional cannabinoid, a terpene, a flavonoid, and an aroma- and flavor-conferring volatile.

5. The fast-acting oral formulation of claim 4 wherein the additional cannabinoid is selected from: Δ8-tetrahydrocannabinol (Δ8-THC), Δ11-tetrahydrocannabinol (Δ11-THC), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA).

6. The fast-acting oral formulation of claim 4 wherein the terpene is selected from: β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, sabinene and phellandrene.

7. The fast-acting oral formulation of claim 4 wherein the flavonoid is selected from: cannaflavin A, cannaflavin B, cannaflavin C, vitexin, isovitexin, apigenin, kaempferol, quercetin, luteolin, cinnamaldehyde, and orientin.

8. The fast-acting oral formulation of claim 4 wherein the aroma- and flavor-conferring molecule is selected from: 2-heptanone, methyl heptanoate, methyl salicylate, methyl anthranilate, and hexanal.

9. A fast-acting oral formulation comprising
(i) one or more of THC, CBD, and/or analogs thereof,
(ii) one or more entourage-restoring molecules and
(iii) a carrier,
wherein the THC, CBD, and/or analogs thereof and the one or more entourage-restoring molecules are provided at ratios that mimic their natural ratios within a cannabis strain.

10. The fast-acting oral formulation of claim 9 comprising THC and CBD.

11. The fast-acting oral formulation of claim 10 wherein the ratio of THC:CBD is between 0.01-100:1.

12. The fast-acting oral formulation of claim 9 wherein the entourage-restoring molecules are selected from one or more of additional cannabinoids, terpenes, flavonoids, and aroma and flavor conferring volatiles.

13. The fast-acting oral formulation of claim 12 wherein the additional cannabinoids are selected from one or more of Δ8-tetrahydrocannabinol (Δ8-THC), Δ11-tetrahydrocannabinol (Δ11-THC), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA).

14. The fast-acting oral formulation of claim 12 wherein the terpenes are selected from one or more of β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, sabinene and phellandrene.

15. The fast-acting oral formulation of claim 12 wherein the flavonoids are selected from one or more of cannaflavin A, cannaflavin B, cannaflavin C, vitexin, isovitexin, apigenin, kaempferol, quercetin, luteolin, cinnamaldehyde, and orientin.

16. The fast-acting oral formulation of claim 12 wherein the aroma and flavor conferring molecules are selected from one or more of 2-heptanone, methyl heptanoate, methyl salicylate, methyl anthranilate, and hexanal.

17. The fast-acting oral formulation of claim 9 wherein the one or more entourage-restoring molecules are each present in the formulation at a ratio of 0.1-100:1 entourage-restoring molecule: THC and/or CBD.

18. The fast-acting oral formulation of claim 9 wherein the carrier comprises an N-acylated fatty amino acid or a salt thereof.

19. The fast-acting oral formulation of claim 18 wherein the N-acylated fatty amino acid comprises one or more of Compounds I-XXXV (FIG. 2), or Compounds α-r (FIG. 3).

20. The fast-acting oral formulation of claim 18, wherein the N-acylated fatty amino acid is selected from monosodium-N-salicyloyl-8-aminocaprylate, disodium-N-salicyloyl-8-aminocaprylate, and N-(salicyloyl)-8- aminocaprylic acid.

21. The fast-acting oral formulation of claim 18, wherein the N-acylated fatty amino acid or a salt thereof comprises 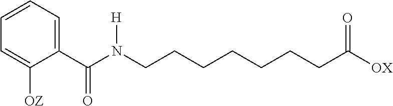
wherein X and Z are independently H, a monovalent cation, a divalent metal cation, or an organic cation.

22. The fast-acting oral formulation of claim 21, wherein X is H.

23. The fast-acting oral formulation of claim 21, wherein X is a monovalent cation comprising sodium or potassium.

24. The fast-acting oral formulation of claim 21, wherein X is a divalent metal cation comprising calcium or magnesium.

25. The fast-acting oral formulation of claim 21, wherein X is an organic cation comprising ammonium or tetramethylammonium.

26. The fast-acting oral formulation of claim 21, wherein Z is H.

27. The fast-acting oral formulation of claim 21, wherein Z is a monovalent cation comprising sodium or potassium.

28. The fast-acting oral formulation of claim 21, wherein Z is a divalent cation comprising calcium or magnesium.

29. The fast-acting oral formulation of claim 21 wherein X is H and Z is H.

30. The fast-acting oral formulation of claim 21, wherein X is H and Z is sodium.

31. The fast-acting oral formulation of claim 21, wherein X is sodium and Z is sodium.

32. The fast-acting oral formulation of claim 18, wherein the N-acylated fatty amino acid is at a dose of 100-200 mg.

33. The fast-acting oral formulation of claim 18, wherein the N-acylated fatty amino acid or salt thereof is at a dose concentration of 100 mg/mL to 300 mg/mL.

34. The fast-acting oral formulation of claim 18, wherein the N-acylated fatty amino acid or salt thereof is at a dose concentration of 250 mg/mL.

35. The fast-acting oral formulation of claim 18, wherein the N-acylated fatty amino acid or salt thereof is at a dose of one to one hundred times the dose of the one or more cannabinoids.

36. The fast-acting oral formulation of claim 9 further comprising a surfactant, detergent, azone, pyrrolidone, glycol or bile salt.

37. The fast-acting oral formulation of claim 9, wherein the formulation comprises one or more plant extracts.

38. The fast-acting oral formulation of claim 9, wherein the formulation is swallowable or chewable.

39. The fast-acting oral formulation of claim 9, wherein the formulation is liquid or solid.

40. The fast-acting oral formulation of claim 9, wherein the formulation is a solution, suspension, gel, juice, oil, paste, emulsion, tincture or spray.

41. The fast-acting oral formulation of claim 9, wherein the formulation is a tablet, capsule, edible, pill, gelcap, granule, gum or sachet.

42. The fast-acting oral formulation of claim 9, wherein the formulation is flavored.

43. The fast-acting oral formulation of claim 9 comprising an effective amount of the formulation.

44. The fast-acting oral formulation of claim 43, wherein the effective amount is a therapeutic amount, a prophylactic amount, a research effective amount, or a recreationally effective amount.

45. The fast-acting oral formulation of claim 43, wherein the effective amount comprises 0.1 mg-100 mg THC.

46. The fast-acting oral formulation of claim 43, wherein the effective amount comprises 0.1 mg-100 mg CBD.

47. A nutritional supplement comprising a formulation of claim 9 and i) a vitamin or a mineral, or ii) a vitamin and a mineral.

48. The nutritional supplement of claim 47 wherein the vitamin comprises Vitamin A, Vitamin B1, Vitamin B6, Vitamin B12, Vitamin C, Vitamin D, Vitamin E, or Vitamin K.

49. The nutritional supplement of claim 47 wherein the mineral comprises calcium, chromium, iodine, iron, magnesium, selenium or zinc.

50. A method of preparing a composition comprising (i) THC and/or CBD and (ii) one or more entourage-restoring molecules, wherein the method comprises adding an absorption enhancer to the composition and wherein the composition has a faster onset of action than an equivalent composition without an absorption enhancer.

51. The method of claim 50, wherein the absorption enhancer is an N-acylated fatty amino acid or a salt thereof.

52. The method of claim 51, wherein the N-acylated fatty amino acid is selected from monosodium-N-salicyloyl-8-aminocaprylate, disodium-N-salicyloyl-8-aminocaprylate, and N-(salicyloyl)-8-aminocaprylic acid.

53. The method of claim 51, wherein the N-acylated fatty amino acid or a salt thereof comprises 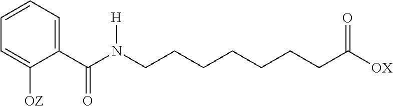
wherein X and Z are independently H, a monovalent cation, a divalent metal cation, or an organic cation.

54. The method of claim 53, wherein X is H.

55. The method of claim 53, wherein X is a monovalent cation comprising sodium or potassium.

56. The method of claim 53, wherein X is a divalent metal cation comprising calcium or magnesium.

57. The method of claim 53, wherein X is an organic cation comprising ammonium or tetramethylammonium.

58. The method of claim 53, wherein Z is H.

59. The method of claim 53, wherein Z is a monovalent cation comprising sodium or potassium.

60. The method of claim 53, wherein Z is a divalent cation comprising calcium or magnesium.

61. The method of claim 53, wherein X is H and Z is H.

62. The method of claim 53, wherein X is H and Z is sodium.

63. The method of claim 53, wherein X is sodium and Z is sodium.

64. The method of claim 53, wherein the N-acylated fatty amino acid provides an administration benefit.

65. The method of claim 53, wherein the administration benefit is a dose-dependent administration benefit.

66. The method of claim 65, wherein the dose-dependent administration benefit is at a dose of 100- 200 mg.

67. The method of claim 65, wherein the dose-dependent administration benefit is at a dose concentration of 100 mg/mL to 300 mg/mL N-acylated fatty amino acid or salt thereof.

68. The method of claim 65, wherein the dose-dependent administration benefit is at a dose concentration of 1-500 mg/mL N-acylated fatty amino acid or salt thereof.

69. The method of claim 68, wherein the dose-dependent administration benefit is at a dose concentration of 250 mg/mL N-acylated fatty amino acid or salt thereof.

70. The method of claim 65, wherein the dose-dependent administration benefit of the N-acylated fatty amino acid or salt thereof is at a dose of one to one hundred times the dose of the one or more synthetic cannabinoids.

71. A method of treating a subject in need thereof comprising administering a therapeutically effective amount of a formulation of claim 9 to the subject thereby treating the subject in need thereof.

72. The method of claim 71, wherein the therapeutically effective amount provides an effective amount, a prophylactic treatment, and/or a therapeutic treatment.

73. A method of reducing or eliminating one or more symptoms of a disease or disorder in a human subject,
wherein said method comprises delivering a therapeutically effective amount of a formulation of claim 9 to the subject, thereby reducing or eliminating one or more symptoms of the disease or disorder, and
wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.
